(19) United States
(12) Patent Application Publication
(10) Pub. No.: US 2007/0021352 A1
(43) Pub. Date: Jan. 25, 2007

(54) PREVENTION AND TREATMENT OF HEARING DISORDERS

(75) Inventors: Jeffery J. Anderson, San Diego, CA (US); Srinivas G. Rao, Encinitas, CA (US); Jay D. Kranzler, La Jolla, CA (US)

Correspondence Address: WILSON SONSINI GOODRICH & ROSATI, 650 PAGE MILL ROAD, PALO ALTO, CA, 94304-1050, US

(73) Assignee: Cypress Bioscience, Inc.

(21) Appl. No.: 11/458,654

(22) Filed: Jul. 19, 2006

Related U.S. Application Data
(60) Provisional application No. 60/700,959, filed on Jul. 20, 2005.

(51) Int. Cl. A61K 38/05; A61K 31/519; A61K 31/425; A61K 31/205; A61K 31/198

(52) U.S. Cl. 514/18; 514/373; 514/262.1; 514/554; 514/562; 514/563; 514/662

(57) ABSTRACT

Compositions, and methods of use thereof, are provided for the prevention, treatment or alleviation of symptoms of hearing are provided. Embodiments of the methods employ zonisamide as the sole active pharmaceutical agent or a combination of zonisamide and another pharmaceutical agent, such as an antioxidant, a NMDA antagonist, an SSRI or a combined SSRI/NMDA antagonist agent. Other embodiments of the method involve the use of zonisamide alone or in combination with another API to prevent, treat or ameliorate one or more symptoms of hearing loss. Hearing disorders treatable with the invention include noise-induced hearing loss, drug-induced hearing loss, central auditory hearing disorder (CAPD), tinnitus and presbyacusis.

### CROSS-REFERENCE

This application claims the benefit of U.S. Provisional Application No. 60/700,959, filed Jul. 20, 2005. This provisional application is expressly incorporated herein in its entirety.

### FIELD OF THE INVENTION

This invention generally relates to methods and compositions for the pharmacological prevention and treatment of hearing disorders, such as hearing loss and tinnitus.

### BACKGROUND OF THE INVENTION

Hearing loss is a growing problem in the industrialized word, with varied and complex etiology. While some forms of hearing loss are clearly genetic in origin, others are either wholly or at least partially environmental in nature. Central, peripheral, or both types of, mechanisms can be involved in hearing loss. Among the forms of hearing loss are presbycusis and noise-induced hearing loss.

Centrally, the decrease in neurotransmitters, such as serotonin (5HT) can lead to hearing loss. Cruz et al. have presented evidence that suggests that the use of citalopram can have a positive impact on auditory processes in the elderly. Oswaldo Laercio M. Cruz et al, Serotonin Reuptake Inhibitors in Auditory Processing Disorders in Elderly Patients: Preliminary Results, Laryngoscope, 114, September 2004, 1656-1659. However, Cruz et al. does not suggest combining S-citalopram with zonisamide, either in a single dosage form or in a treatment regime.

Presbycusis (also presbyacusis) begins after age 20 but is usually significant only in persons over 65. Men are affected more often and more severely than women. Stiffening of the basilar membrane and deterioration of the hair cells, stria vascularis, ganglion cells, and cochlear nuclei may play a role in pathogenesis, and presbycusis appears to be related in part to noise exposure. It first affects the highest frequencies (18-20 kHz) and gradually begins to affect the 4- to 8-kHz range by age 55 to 65, although variation is considerable. Some persons are severely handicapped by age 60, and some are essentially untouched by age 90. The loss of high-frequency hearing makes discrimination of speech particularly difficult. Thus, many persons who have this type of hearing loss have difficulty understanding conversation, particularly when background noise is present, and complain that others mumble. Although speech reading (lip reading), auditory training for making maximum use of non-auditory clues, and amplification with a hearing aid are helpful, better therapeutic alternatives would be welcome. L-carnitine has been administered to rats in a model of presbycusis. A. Derin et al., The Effects of L-carnitine on Presbyacusis in the Rat Model, Clin. Otolaryngol., 2004, 29, 238-241. However, Derin et al. do not teach or suggest the combination of zonisamide and L-carnitine, either in compositions or in methods relating to hearing loss.

Noise-induced hearing loss can arise under either acute or chronic circumstances. Extremely loud sounds can give rise to sudden hearing loss. While such hearing loss not infrequent, hearing loss due to long-term exposure to excessive noise is more common.

Noise-induced hearing loss can give rise to multifarious problems. In addition to the inability to hear certain sounds, especially in the upper registers, one experiencing such hearing loss may also experience tinnitus, or ringing in the ears, which is characterized by abnormal sounds and auditory sensations that may persist for various lengths of time after cessation of auditory stimulation. Additionally, noise can mechanically irritate the inner ear, giving rise to an inflammatory response characterized by fluid buildup and concomitant dampening of sound transmission within the ear. Moreover, excessive noise can cause damage to the 8^{th} nerve, giving rise to a neuronal type of hearing loss. In the earlier stages of neuronal hearing loss, the patient experiences a degradation of his ability to process sounds and speech. Thus, for instance, the patient may lose the ability to discriminate between certain words or to understand certain persons, especially those whose voices are in the upper or lower registers.

Another important type of hearing loss is drug-induced hearing loss. Ototoxic drugs include chemotherapeutic agents, such as antineoplastic agents and antibiotics. Other ototoxic drugs include loop-diuretics, quinines or a quinine-like compound, and salicylate or salicylate-like compounds.

Aminoglycosides are antibiotics that have been used for the treatment of Gram-negative bacterial infections and some aerobic Gram-positive bacterial infections. Despite their utility, however, they have serious side effects, including ototoxicity. Aminoglycoside ototoxicity is associated with the destruction of the sensory hair cells in organ of Corti of the cochlea of the inner ear. See Bates et al., “Aminoglycoside Ototoxicity,” Drugs of Today 39(4), 277-285 (2003)(incorporated herein by reference in its entirety).

Cisplatin is an antineoplastic agent that is commonly used in the treatment of cancer. Like many antineoplastic agents, however, cisplatin has several known and widely documented toxicities, including cytotoxicity. In particular, cisplatin treatment gives rise to hair cell degeneration in the organ of Corti. Although D-methionine has been suggested as a protectant for the cochlea during administration of cisplatin, effective methods of protecting against cisplatin-induced hearing loss have yet to be fully realized. See Campbell et al., “D-Methionine provides excellent protection from cisplatin ototoxicity in the rat,” Hearing Research 102, 90-98 (1996)(incorporated herein by reference in its entirety). Moreover, D-methionine is of extremely low potency.

Central auditory processing disorder (CAPD) is a deficit in the neural processing of auditory stimuli that is not due to higher order language, cognitive or related factors. While higher order cognitive-communication or language-related functions may be associated with CAPD, they are not included within the definition of CAPD. Nevertheless, CAPD may exacerbate or even give rise to one or more higher order cognitive-communication or language-related function disorders. Indeed, CAPD can lead to, or be associated with, one or more difficulties in learning, speech, language, social functioning. At present, there are no accepted pharmaceutical therapeutic approaches to treatment of CAPD.

There is a need for pharmaceuticals and methods for protecting the ear from damage by excessive noise. Additionally, there is a need for compositions and methods of treating a mammal to reduce, ameliorate or counteract one or more symptoms of noise-induced hearing loss, such as the abnormal sounds and auditory sensations associated with tinnitus. Additionally, there is a need for pharmaceuticals and methods of treating a mammal, such as a human, to restore hearing to the mammal by treating the effects of noise-induced hearing loss.

There is likewise a need for pharmaceuticals and methods for protecting the ear from damage by an ototoxic drug. Additionally, there is a need for compositions and methods of treating a mammal to reduce, ameliorate or counteract one or more symptoms of drug-induced hearing loss. Additionally, there is a need for pharmaceuticals and methods of treating a mammal, such as a human, to restore hearing to the mammal by treating the effects of drug-induced hearing loss.

### SUMMARY OF THE INVENTION

The foregoing and other needs are met by embodiments of the invention, which provide a method of preventing or treating a hearing disorder in a mammal, such as a human. The method includes administering to the mammal an amount of zonisamide, either alone or in combination with one or more active pharmaceutical ingredients, sufficient to prevent or treat one or more hearing disorders. The additional pharmaceutical ingredients useful in combination with zonisamide include antioxidants or spin trapping agents, NMDA antagonists, agents combining SSRI and NMDA antagonist activity and combinations of one or more SSRIs and one or more NMDA antagonists.

The foregoing and other needs are further met by embodiments of the invention, which provide compositions for preventing one or more hearing disorders in a mammal, such as a human. The compositions of the invention comprise zonisamide alone in combination with a second active pharmaceutical ingredient in an amount sufficient to provide hearing protective or hearing loss treating benefit to a mammal, such as a human.

The foregoing and other needs are further met by embodiments of the invention, which provide method of preventing or treating a hearing disorder in a mammal, such as a human, comprising administering to the mammal a therapeutic amount of a composition comprising a compound having dopamine releasing and NMDA antagonist activity. The invention further provides dosage forms, including mixtures, comprising a compound having both dopamine releasing and NMDA antagonist activity.

The foregoing and other needs are further met by embodiments of the invention, which provide a method of preventing or treating a hearing disorder in a mammal, comprising administering to the mammal a therapeutic amount of a composition comprising a compound having acetylcholine release inducing, antioxidant, NMDA antagonist and norepinephrine reuptake inhibiting activity. The invention further provides dosage forms, including mixtures, comprising a compound having acetylcholine release inducing, antioxidant, NMDA antagonist and norepinephrine reuptake inhibiting activity.

The foregoing and other needs are further met by embodiments of the invention, which provide a method of preventing or treating a hearing disorder in a mammal, comprising administering to the mammal a therapeutic amount of a composition comprising a compound having monamineoxidase-A inhibiting, serotonin reuptake inhibiting and antioxidant activity.

The foregoing and other needs are further met by embodiments of the invention, which provide a method of preventing or treating a hearing disorder in a mammal, comprising administering to the mammal a therapeutic amount of a composition comprising at least a first compound having calcium channel blocking activity and a second compound having selective serotonin reuptake inhibiting activity, norepinephrine-serotonin reuptake inhibiting activity or monamineoxidase-A inhibiting activity.

The foregoing and other needs are further met by embodiments of the invention, which provide a method of preventing or treating a hearing disorder in a mammal, comprising administering to the mammal a therapeutic amount of a composition comprising at least a first compound having 5HT reuptake inhibiting, norepinephrine reuptake inhibiting, acetylcholine releasing and N-methyl-D-aspartate antagonistic activity.

The foregoing and other needs are further met by kits containing two or more active pharmaceutical ingredients for the treatment or prevention of hearing disorders in separate dosage forms.

### DETAILED DESCRIPTION OF THE INVENTION

The invention provides compositions, methods and kits for prevention or treatment of hearing disorders. In the context of this invention, the conjunction “or,” unless otherwise qualified, is used in the inclusive sense. Thus, for example, “prevention or treatment” means “prevention, treatment or both.”

A “hearing disorder” is a central or peripheral hearing disorder, such as hearing loss, tinnitus or hyperacusis. Hearing loss includes conductive hearing loss and sensorineural hearing loss. Types of sensorineural hearing loss include: presbycusis, noise-induced hearing loss (NIHL) and drug-induced hearing loss.

Prevention of a hearing disorder means providing partial or complete protection against a hearing disorder. Thus, prevention of a hearing disorder includes provision of partial or complete protection against hearing loss, such as noise-induced hearing loss or drug-induced hearing loss, tinnitus or hyperacusis.

Treatment of a hearing disorder means providing palliation, amelioration or reversal of a hearing disorder. Thus, treatment of a hearing disorder includes provision of partial or complete palliation, amelioration or reversal of hearing loss, tinnitus or hyperacusis.

Within the context of the present invention, prevention of hearing loss means protection against hearing loss, such as noise-induced hearing loss, drug-induced hearing loss, central auditory processing disorder (CAPD), tinnitus or presbyacusis. In particular, prevention of hearing loss includes protection against damage to hair cells within the cochlea of the inner ear. By protecting the hair cells from noise- or drug-induced damage, the invention prevents loss of hearing by the patient.

Within the context of the invention, treatment of hearing loss means reducing hearing loss or ameliorating one or more symptoms of hearing loss, especially noise-induced hearing loss, drug-induced hearing loss, and CAPD. Thus, treatment of hearing loss includes: improving hearing; reducing transmission of abnormal sounds and auditory sensations associated with tinnitus; reducing fluid accumulation associated with disorders of the inner ear; facilitating central auditory processing of sounds in the inner ear; improving voice recognition or processing; and/or ameliorating one or more additional symptoms of hearing loss.

When used herein, the term patient means a mammal to which one or more zonisamide-containing compositions for the prevention or treatment of hearing loss is administered in order to elicit a therapeutic effect. A therapeutic effect in this sense includes prophylaxis, treatment and/or both. The person skilled in the art will recognize that the therapeutic effect will vary depending upon the desired outcome of administering the zonisamide-containing compositions to the patient.

The invention provides compositions for protection against hearing loss, treatment of hearing loss, or both. In some embodiments, the compositions comprise zonisamide as the sole active pharmaceutical ingredient. In such embodiments, zonisamide may be admixed with one or more inactive ingredients. Zonisamide may also be coated with one or more inactive ingredients. In some embodiments, zonisamide may be mixed with one or more other inactive ingredients and coated with one or more inactive ingredients. In some embodiments, zonisamide and at least one inactive ingredient may be separated (segregated) from one another to prevent their admixture. Such segregation may be effected in order to ensure release of one ingredient before the other, to prevent reaction between the ingredients or both. Inactive ingredients useful in various embodiments of the invention are discussed in more detail below.

In other embodiments, zonisamide is combined with another active pharmaceutical ingredient in the same dosage form. In this context “combined” means that zonisamide and the other active pharmaceutical ingredient are together in the same dosage form, such that they may be transported, dispensed and taken by the patient in a single convenient dosage form. Exemplary dosage forms are discussed in more detail below.

In such embodiments, zonisamide may be admixed with one or more active pharmaceutical ingredients. Zonisamide may also be coated with one or more active pharmaceutical ingredients. In some embodiments, zonisamide may be mixed with one or more other active pharmaceutical ingredients and coated with one or more active pharmaceutical ingredients. In some embodiments, zonisamide and at least one active pharmaceutical ingredient may be separated (segregated) from one another to prevent their admixture. Such segregation may be effected in order to ensure release of one ingredient before the other, to prevent reaction between the ingredients or both. Active pharmaceutical ingredients useful in various embodiments of the invention are discussed in more detail below.

I. Compositions

Compositions for the treatment of hearing disorders comprise one or more active pharmaceutical ingredients (APIs). The person skilled in the art will recognize that a reference to a particular compound, unless otherwise modified, embraces as well pharmaceutically acceptable salts or polymorphs thereof. In other words, unless it is specified that a pharmaceutical composition contains only base compound, reference to a compound name is intended to embrace pharmaceutically acceptable salts and polymorphs.

In addition, the person skilled in the art will recognize that, where a referenced compound has one or more chiral centers, reference to the compound without further modification generically embraces each of the stereoisomers, if any exist, as well. Methods of separating stereoisomers are known in the art. Thus, unless it is specified that a pharmaceutical composition contains a racemate or a specific stereoisomer of a compound, reference to the compound name is intended to generically embrace the racemate, each stereoisomer individually, and mixtures of two or more stereoisomers, if any exist.

A. APIs Useful for Treatment of Hearing Disorders

1. Zonisamide

Zonisamide (1,2-benzisoxazole-3-methanesulfonamide) compliments the pharmacology of a norepinephrine-epinephrine reuptake inhibitor (NERI) by: 1) enhancing serotonin (5HT) and dopamine (DA) transmission and 2) by blocking sodium (Na⁺) and calcium (Ca⁺⁺) channels. These actions enhance the efficacy of NERIs in the treatment of depression, schizophrenia, anxiety disorders, sleep-related breathing disorders, snoring, insomnia, migraine headache, chronic tension-type headache, hot flashes, lower back pain, neuropathic pain, functional somatic syndromes and obesity.

2. Antioxidants and/or Spin-Trapping Agents

Antioxidants, such as allopurinol, glutathione, methionine and L-carnitine reduce noise-induced damage to hair cells of the inner ear. These agents bind to or metabolize reactive oxygen species and provide protection against the damage induced by these toxic mediators. Such antioxidants and/or spin-trapping agents include allopurinol, glutathione, L-carnitine, methionine and pharmaceutically acceptable salts, polymorphs and combinations thereof.

a. Allopurinol

Allopurinol or 1,5-dihydro-4H-pyrazolo[3,4-d]pyrimidin-4-one is an antioxidant in the class of compounds known as xanthine oxidase inhibitors and is known as a pharmaceutical for the treatment of hyperuricemia and chronic gout.

b. Glutathione

Glutathione or N-(N-L-γ-glutamyl-L-cysteinyl)glycine is

c. Methionine

Methionine or (S)-2-amino-4-(methylthio)butanoic acid is known as a hepatoprotectant, an antidote for acetaminophen poisoning and as a urinary acidifier.

d. L-Carnitine

L-carnitine, or 3-hydroxy-4N-trimethylaminobutryic acid, is a biological isomer of carnitine, which is a non-toxic compound that has an equal LD₅₀ value with other amino acids. It is known to stimulate fatty acid oxidation in the liver, heart and skeletal muscles. Carnitine is also a scavenger of superoxide ion and decreases free radical synthesis by inhibiting xanthine oxidase activity. Carnitine is known to have other effects in biological systems. For example, carnitine plays a role in the active transportation of long-chain fatty acids into the mitochondrial matrix and intramitochondrial acyl-CoA/CoA regulation; hence carnitine is essential for mitochondrial oxidation of long-chain fatty acids. In addition, carnitine increases the proliferative response of human lymphocytes to mitogenic stimulation and polymorphonuclear chemotaxis, stabilizes the cell membrane and increases Ca²⁺ transport.

e. Combinations of Antioxidants

Combinations of antioxidants include: allopurinol and glutathione; glutathione and methionine; allopurinol and methionine; and allopurinol, glutathione and methionine.

3. NMDA Antagonists

N-methyl-D-aspartate (NMDA) antagonists, such as riluzole, caroverine, memantine, magnesium or mixtures thereof, block excitotoxic actions of glutamate within the inner ear. Glutamate is a mediator of noise-induced damage to the hair cells of the inner ear. Thus, blocking NMDA receptors provides protection against the toxic effects of glutamate.

a. Riluzole

Riluzole, or 2-amino-6-trofluoromethoxybenzothiazole, is a NMDA antagonist having wide-spectrum neuroprotective activity. It is also an anti-convulsant, which also has anti-ischemic and sedative properties. J. Wang et al., Riluzole Rescues Cochlear Sensory Cells From Acoustic Trauma in the Guinea Pig, Neuroscience, 111 (3), 2002, 635-648. Wang et al. have shown in a Guinea pig model that perfusion of riluzole into the cochlea via an osmotic minipump prevents mitochondrial damage and subsequent translocation of cytochrome c, DNA fragmentation and hair cell degeneration. While Wang et al. failed to demonstrate oral dosing with riluzole, let alone combination therapy with riluzole and zonisamide, they did show that perfusion with riluzole can rescue the cochlea of Guinea pigs from noise-induced trauma.

b. Caroverine

Caroverine, or 1-[2-(diethylamino)ethyl]-3-[(4-methoxyphenyl)methyl]-2-(1H)-quinoxalinone, is an NMDA antagonist and is known to be useful as an antispasmotic. Caroverine is an antagonist of two glutamate receptors. Zhiquiang Chen et al., Protection of Auditory Function Against Noise Trauma with Local Caroverine Administration in Guinea Pigs, Hearing Research, 197 (2004), 131-136. Chen et al. have presented data that demonstrate protection of cochlea from noise trauma in a guinea pig model. Thus, it is expected that combination therapy with zonisamide and caroverine will protect the cochlea from noise trauma. As caroverine is an NMDA antagonist, it is expected that other NMDA antagonists will work together with zonisamide to protect a mammal, such as a human, from noise-induced hearing loss.

c. Memantine

Memantine or 3,5-dimethyltricyclo[3.3.1.1]decan-1-amine is an NMDA antagonist and is know to be useful as a muscle relaxant.

d. Magnesium

Magnesium (Mg²⁺) is an essential factor in regulating cellular membrane permeability, neuromuscular excitability and energy production. Magnesium has been shown to antagonize the N-methyl-D-aspartate receptor. Daily ingestion of 122 mg of magnesium for 10 days was shown to protect against noise-induced temporary threshold shift. Attias et al., Reduction in noise-induced temporary threshold shift in humans following oral magnesium intake, Clin. Otolaryngol., 2004, 29, 635-641, which is incorporated herein by reference. Daily ingestion of 6.7 mmol of magnesium aspartate showed a significant effect on permanent threshold shift as compared to placebo (sodium aspartate) in normal, healthy volunteers exposed to rifle noise six days per week for eight weeks. Attias et al., Oral Magnesium Intake Reduces Permanent Hearing Loss Induced by Noise Exposure, Am. J. Otolaryngology, Vo. 15, no 1, (Jan-Feb), 1994, 26-32, which is incorporated herein by reference. Magnesium has also been used to treat sudden sensorineural hearing loss (SSNHL). Nageris et al., Magnesium Treatment for Sudden Hearing Loss, Ann. Otol. Rhinol. Laryngol. 113, 2004, 672-675, which is incorporated by reference.

e. Combinations of NMDA Antagonists

Combinations of NMDA antagonists include: riluzole and caroverine; caroverine and memantine; riluzole and memantine; riluzole, caroverine and memantine; riluzole and magnesium; caroverine and magnesium, memantine and magnesium; riluzole, caroverine and magnesium; caroverine, memantine and magnesium; riluzole, memantine and magnesium; and riluzole, caroverine, memantine and magnesium.

4. SSRI/NMDA Antagonists

Selective serotonin reuptake inhibitors (SSRIs) enhance synaptic levels of serotonin in the brain and enhance hearing by improving auditory processing, increasing the signal: noise ratio of environmental sounds, and by heightening attention. Examples of SSRIs include fluoxetine, sertraline, S-citalopram and combinations thereof. SSRIs may be combined with NMDA antagonists, such as the aforementioned riluzole, caroverine, memantine and combinations thereof. Additionally, there are SSRIs with both SSRI and NMDA antagonist activity, such as alaproclate (2-(p-chlorophenyl)-1,1-dimethyl-2-aminopropanoate).

a. Fluoxetine

Fluoxetine, or (±)-N-methyl-γ-[4-trifluoromethyl)-phenoxy]benzenepropanamine, is an SSRI known to be an antidepressant.

b. Sertraline

Sertraline, or (1S-cis)-4-(3,4-diclorophenyl)-1,2,3,4-tetrahydro-N-methyl-1-naphthalenamine, is an SSRI known to be an antidepressant.

c. S-Citalopram

S-citolopram, or 1-[3-(dimethylamino)propyl]-1-(4-fluorophenyl)-1,3-dihydro-5-isobenzofurancarbonitrile, is an SSIR known to have antidepressant activity. Cruz et al. have presented evidence that suggests that the use of citalopram can have a positive impact on auditory processes in the elderly. Oswaldo Laercio M. Cruz et al, Serotonin Reuptake Inhibitors in Auditory Processing Disorders in Elderly Patients: Preliminary Results, Laryngoscope, 114, September 2004, 1656-1659.

d. Alaproclate

Alaproclate ([2-(p-chlorophenyl)-1,1-dimethyl-2-aminopropanoate]) is a known antidepressant having both selective serotonin reuptake inhibiting and N-methyl-D-aspartate antagonist activity.

e. SSRI and NMDA Antagonist Combinations

Combinations of SSRI and NMDA antagonists include: one or more SSRI selected from the group consisting of fluoxetine, sertraline and S-citolopram with one or more NMDA antagonists selected from the group consisting of riluzole, caroverine, memantine and magnesium. Particular combinations include; fluoxetine and riluzole; fluoxetine and caroverine; fluoxetine and memantine; fluoxetine and magnesium; sertraline and riluzole; sertraline and caroverine; sertraline and memantine; sertraline and magnesium; S-citolopram and riluzole; S-citolopram and caroverine; S-citolopram and memantine; S-citolopram and magnesium; fluoxetine and sertraline with a NMDA antagonist (such as riluzole, caroverine, memantine; magensium and combinations of two or more thereof) fluoxetine and S-citolopram and a NMDA antagonist (such as riluzole, caroverine, memantine, magnesium and combinations of two or more thereof); sertraline and S-citolopram and a NMDA antagonist (such as riluzole, caroverine, memantine, magnesium and combinations of two or more thereof); fluoxetine, sertraline and S-citolopram and a NMDA antagonist (such as riluzole, caroverine, memantine, magnesium and combinations of two or more thereof).

5. Dopamine Releaser/NMDA Antagonists

A. Amantadine

Amantadine (tricycle[3.3.1.1^{3,7}]decan-1-amine) has combined dopamine releasing and N-methyl-D-aspartate antagonistic activity. It has been used for the treatment of Parkinsonism, as an antiviral in the treatment of influenza A and for drug-induced extra-pyramidal reactions. In theory, the dopamine releasing effect of amantadine enhances central auditory processing, while the NMDA antagonistic effect protects the inner ear hair cells from glutamate-induced toxicity.

B. Combinations

A compound having combined dopamine releasing and NMDA antagonistic activity, such as amantadine, can be combined with one or more additional for the prevention or treatment of hearing disorders. Such additional compounds can act in coordination with the dopamine releasing/NMDA antagonist compound. In some embodiments, the additional compounds enhance the hearing disorder protecting or treating activity of the dopamine releaser/NMDA antagonist, allowing treatment at a lower dose of the dopamine releaser/NMDA antagonist, or allowing treatment of patients who would not respond to dopamine releaser/NMDA antagonist therapy at a dose below its toxic dose. In other embodiments, the additional compound or compounds ameliorate one or more undesirable side effects of the dopamine releaser/NMDA antagonist.

In particular embodiments, a compound having combined dopamine releasing and NMDA antagonistic activity, such as amantadine, can be combined with one or more additional active pharmaceutical ingredients, such as one or more member of the group consisting of zonisamide, selective serotonin reuptake inhibitors, and antioxidants. Thus, particular combinations possible within the scope of this invention include: amantadine and zonisamide; amantadine and a SSRI; amantadine and an antioxidant; amantadine, zonisamide and a SSRI; amantadine, zonisamide and an antioxidant; amantadine, a SSRI and an antioxidant; and amantadine, zonisamide, a SSRI and an antioxidant. Particular SSRIs and antioxidants are described in more detail above.

6. Acetylcholine Release Inducer/Antioxidant/NMDA Antagonist/NERI

Compounds having combined acetylcholinesterase inducing, antioxidant, N-methyl-D-aspartate antagonist and norepinephrine reuptake inhibiting activity, such as bifemelane, can be used in the prevention or treatment of hearing disorders, such as hearing loss and tinnitus. In theory, compounds such as bifemelane enhance brain levels of acetylcholine and norepinephrine, thereby improving auditory processing, speech recognition and hearing perception. Also in theory, compounds such as bifemelane, by blocking NMDA receptors and by acting as antioxidants, provide protection to the inner ear hair cells.

Bifemelane (N-methyl4-[2-(phenylmethyl)phenoxy]-1-butanamine) is a compound combining acetylcholinesterase inducing, antioxidant, N-methyl-D-aspartate antagonist and norepinephrine reuptake inhibiting activity that has been used as a nootropic agent.

7. MAO-A Inhibitor/SRI/Antioxidant

Compounds having combined monamineoxidase-A inhibiting, serotonin reuptake inhibiting and antioxidant activity, such as pirlindole, can be used alone or in combination with an NMDA antagonist or amantadine for the treatment of a hearing disorder, such as hearing loss or tinnitus. In theory, the central effects of compounds such as pirlindole via increasing norepinephrine and serotonin increase auditory processing, while the antioxidant effect of compounds such as pirlindole protects inner ear hair cells from damage induced by oxidative species.

8. Norepinephrine and Serotonin Reuptake Inhibitor/Weak NMDA Antagonists

Norepinephrine and serotonin reuptake inhibitor/weak NMDA antagonists, such as milnacipran and bicifadine, can be administered along with zonisamide to provide central and peripheral treatment effects. The central effects of such compounds are through the norepinephrine reuptake inhibition and serotonin reuptake inhibition activities, which provide improved auditory processing. The peripheral effects include protection of inner ear hair cells via the N-methyl-D-aspartate antagonist activity.

a. Milnacipran

Milnacipran (cis-(±)-2-(aminomethyl)-N,N-diethyl-1-phenylcyclopropanecarboxamide) is a known antidepressant having norepinephrine reuptake and serotonin reuptake inhibiting effects. Milnacipran is also a weak N-methyl-D-aspartate antagonist. In some embodiments of the invention, zonisamide is used in combination with milnacipran. In particular embodiments, milnacipran and zonisamide are administered in separate dosage forms. In other embodiments, zonisamide and milnacipran are combined in the same dosage form, for example in a common capsule or tablet. In particular embodiments, separate zonisamide and milnacipran dosage forms are combined in a kit, such as a blister pack, as discussed in more detail below.

b. Bicifadine

Bicifadine (1-(4-methylphenyl)-3-azabicyclo[3.1.0]hexane) is a non-opioid analgesic having norepinephrine reuptake inhibiting, serotonin reuptake inhibiting and weak NMDA antagonistic activities. In some embodiments of the invention, zonisamide is used in combination with bicifadine. In particular embodiments, bicifadine and zonisamide are administered in separate dosage forms. In other embodiments, zonisamide and bicifadine can be combined in the same dosage form, for example in a common capsule or tablet. In particular embodiments, separate zonisamide and bicifadine dosage forms are combined in a kit, such as a blister pack, as discussed in more detail below.

9. Calcium Channel Antagonists and SSRI or NSRI

In some embodiments, a calcium channel antagonist, such as nimodipine or verapamil, is used in combination with a selective serotonin reuptake inhibitor or a norepinephrine selective reuptake inhibitor in the prevention or treatment of hearing disorders.

a. Nimodipine

Nimodipine (1,4-dihydro-2,6-dimethyl-4-(3-nitrophenyl)-3,5-pyridinedicarboxylic acid 2-methoxyethyl 1-methoxyethyl ester) is a calcium channel antagonist known to have vasodilating activity.

b. Verapamil

Verapamil (α-[3-[[2-(3,4-dimethoxyphenyl)ethyl]methylamino]propyl]-3,4-dimethoxy-α-(1-methylethyl)-benzeneacetonitrile) is a calcium channel antagonist known to have anti-angina and anti-arrhythmic activity.

c. SSRIs

Selective serotonin reuptake inhibitors, such as fluoxetine, sertraline and S-citolopram, block the reuptake of serotonin in the synaptic cleft, thereby providing greater serotonin-dependent neurotransmission.

d. NSRIs

Norepinephrine selective reuptake inhibitors, such as atomoxetine, selectively block the reuptake of norepinephrine in the synaptic cleft, thereby increasing the functional concentration of this neurotransmitter. NSRIs

10. 5HT SRI/NRI/ACE Releaser/NMDA Antagonist

A compound having 5HT serotonin reuptake inhibiting, norepinephrine reuptake inhibiting, acetylcholine releasing and N-methyl-D-aspartate antagonist activity can be used either alone or in addition to an NMDA antagonist for the treatment or prevention of a hearing disorder, such as hearing loss or tinnitus.

a. Indeloxazine

Indeloxazine (2-[1H-inden-7-yloxy)methyl]morpholine hydrochloride) is known to be an antidepressant, nootropic having 5HT serotonin reuptake inhibiting, norepinephrine reuptake inhibiting, acetylcholine releasing and N-methyl-D-aspartate antagonist activity. In some embodiments, indeloxazine is used alone for the treatment or prevention of a hearing disorder, such as hearing loss or tinnitus. In theory, the ability of indeloxazine to increase brain serotonin, norepinephrine and acetylcholine improves central auditory processing, speech recognition and hearing perception, while its NMDA blocking activity provides protection to the inner ear hair cells.

b. Indeloxazine plus NMDA antagonist

In some embodiments, indeloxazine is used together with a compound selected from the group consisting of NMDA antagonists. In theory, the ability of indeloxazine to increase brain serotonin, norepinephrine and acetylcholine improves central auditory processing, speech recognition and hearing perception, while its NMDA blocking activity provides protection to the inner ear hair cells. The additional compound having NMDA antagonist activity provides protection to the inner ear hair cells. NMDA antagonists that may be combined with indeloxazine include riluzole, caroverine, memantine, magnesium and mixtures thereof.

Particular combinations of indeloxazine and an NMDA antagonist include: indeloxazine and riluzole; indeloxazine and caroverine; indeloxazine and memantine; and indeloxazine and magnesium.

B. Salts, Stereoisomers, Polymorphs and Derivatives

Although described above with reference specific to compounds, one can also utilize stereoisomers, polymorphs, metabolites, derivates and salts of the active compounds. Methods for synthesis of these compounds are known to those skilled in the art. Examples of pharmaceutically acceptable salts include, but are not limited to, mineral or organic acid salts of basic residues such as amines, and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Conventional non-toxic salts include those derived from inorganic acids such as hydrochloric, hydrobromic, sulfuric, sulfamic, phosphoric and nitric acid; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic and isethionic acids. The pharmaceutically acceptable salts can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 17th ed. (Mack Publishing Company, Easton, Pa, 1985, p. 1418).

Stereoisomers are compounds made up of the same atoms having the same bond order but having different three-dimensional arrangements of atoms which are not interchangeable. The three-dimensional structures are called configurations. Two kinds of stereoisomers include enantiomers and diastereomers. Enantiomers are two stereoisomers which are non-superimposable mirror images of one another. This property of enantiomers is known as chirality. The terms “racemate”, “racemic mixture” or “racemic modification” refer to a mixture of equal parts of enantiomers. The term “chiral center” refers to a carbon atom to which four different groups are attached. Choice of the appropriate chiral column, eluent, and conditions necessary to effect separation of the pair of enantiomers is well known to one of ordinary skill in the art using standard techniques (see e.g. Jacques, J. et al., “Enantiomers, Racemates, and Resolutions”, John Wiley and Sons, Inc. 1981). Diastereomers are two stereoisomers which are not mirror images but also not superimposable. Diastereoisomers have different physical properties and can be separated from one another easily by taking advantage of these differences.

Different polymorphs of the compounds may also be used. Polymorphs are, by definition, crystals of the same molecule having different physical properties as a result of the order of the molecules in the crystal lattice. The polymorphic behavior of drugs can be of crucial importance in pharmacy and pharmacology. The differences in physical properties exhibited by polymorphs affect pharmaceutical parameters such as storage stability, compressibility and density (important in formulation and product manufacturing), and dissolution rates (an important factor in determining bio-availability). Differences in stability can result from changes in chemical reactivity (e.g. differential oxidation, such that a dosage form discolors more rapidly when comprised of one polymorph than when comprised of another polymorph) or mechanical changes (e.g. tablets crumble on storage as a kinetically favored polymorph converts to thermodynamically more stable polymorph) or both (e.g. tablets of one polymorph are more susceptible to breakdown at high humidity).

A prodrug is a covalently bonded substance which releases the active parent drug in vivo. Prodrugs are prepared by modifying functional groups present in the compound in such a way that the modifications are cleaved, either in routine manipulation or in vivo, to yield the parent compound. Prodrugs include compounds wherein the hydroxy or amino group is bonded to any group that, when the prodrug is administered to a patient, cleaves to form a free hydroxyl or free amino, respectively. Examples of prodrugs include, but are not limited to, acetate, formate and benzoate derivatives of alcohol and amine functional groups.

A metabolite of the above-mentioned compounds results from biochemical processes by which living cells interact with the active parent drug or other formulas or compounds in vivo. Metabolites include products or intermediates from any metabolic pathway.

C. Formulations

1. General Considerations

The compounds, or pharmaceutically acceptable salts thereof, or polymorphic variations thereof, can be formulated as pharmaceutical compositions. Such compositions can be administered orally, buccally, intravenously, parenterally, by inhalation spray, rectally, intradermally, transdermally, pulmonary, nasally or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrastemal injection, or infusion techniques. In the preferred embodiment the composition is administered orally.

Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

The active compounds (or pharmaceutically acceptable salts thereof) may be administered per se or in the form of a pharmaceutical composition wherein the active compound(s) is in admixture or mixture with one or more pharmaceutically acceptable carriers, excipients or diluents. Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, or preservatives.

The compounds may be complexed with other agents as part of their being pharmaceutically formulated. The pharmaceutical compositions may take the form of, for example, tablets or capsules prepared by conventional means with pharmaceutically acceptable excipients such as binding agents (e.g., acacia, methylcellulose, sodium carboxymethylcellulose, polyvinylpyrrolidone (Povidone), hydroxypropyl methylcellulose, sucrose, starch, and ethylcellulose); fillers (e.g., corn starch, gelatin, lactose, acacia, sucrose, microcrystalline cellulose, kaolin, mannitol, dicalcium phosphate, calcium carbonate, sodium chloride, or alginic acid); lubricants (e.g. magnesium stearates, stearic acid, silicone fluid, talc, waxes, oils, and colloidal silica); and disintegrators (e.g. micro-crystalline cellulose, corn starch, sodium starch glycolate and alginic acid. If water-soluble, such formulated complex then may be formulated in an appropriate buffer, for example, phosphate buffered saline or other physiologically compatible solutions. Alternatively, if the resulting complex has poor solubility in aqueous solvents, then it may be formulated with a non-ionic surfactant such as TWEEN™, or polyethylene glycol. Thus, the compounds and their physiologically acceptable solvates may be formulated for administration.

Liquid formulations for oral administration prepared in water or other aqueous vehicles may contain various suspending agents such as methylcellulose, alginates, tragacanth, pectin, kelgin, carrageenan, acacia, polyvinylpyrrolidone, and polyvinyl alcohol. The liquid formulations may also include solutions, emulsions, syrups and elixirs containing, together with the active compound(s), wetting agents, sweeteners, and coloring and flavoring agents. Various liquid and powder formulations can be prepared by conventional methods for inhalation by the patient.

Delayed release and extended release compositions can be prepared. The delayed release/extended release pharmaceutical compositions can be obtained by complexing drug with a pharmaceutically acceptable ion-exchange resin and coating such complexes. The formulations are coated with a substance that will act as a barrier to control the diffusion of the drug from its core complex into the gastrointestinal fluids. Optionally, the formulation is coated with a film of a polymer which is insoluble in the acid environment of the stomach, and soluble in the basic environment of lower GI tract in order to obtain a final dosage form that releases less than 10% of the drug dose within the stomach.

In addition, combinations of immediate release compositions and delayed release/extended release compositions may be formulated together.

In some embodiments, zonisamide is formulated as the sole active pharmaceutical ingredient (API) in a dosage form. Such zonisamide dosage form may be used alone or in combination therapy with one or more additional dosages containing one or more active pharmaceutical ingredients for prevention or treatment of hearing loss. In such embodiments, the daily dosage of zonisamide is conveniently provided in a single dosage form as described herein, or may be divided amongst two, three, four or more dosages. In some embodiments, the dosage of zonisamide is in the range of about 5 to about 250 mg per day. In particular embodiments, the dosage of zonisamide is in the range of about 10 to 100 mg per day. In specific embodiments, the dosage range for zonisamide is from about 25 to about 50 mg per day.

D. Specific Combinations

In some embodiments the invention provides dosage forms combining of Zonisamide and at least one other active pharmaceutical ingredient. In some embodiments, such other active pharmaceutical ingredient is an antioxidant or spin trapping agent, an NMDA antagonist, a SSRI agent, an agent having combined SSRI and NMDA antagonist activity, or combinations (including mixtures) thereof. In some such dosage forms, zonisamide is mixed directly with at least one other active pharmaceutical ingredient. In others, the zonisamide is segregate from at least one other active pharmaceutical ingredient by a coating, a shell, a capsule or some other means for preventing admixture of zonisamide with at least one other active pharmaceutical ingredient, while maintaining both ingredients in the same dosage form.

1. Zonisamide and Antioxidants or Spin Trapping Agents

In some embodiments, zonisamide is combined with one or more active pharmaceutical agents that bind to or metabolize reactive oxygen species and provide protection against the damage induced by oxygen species, which are toxic mediators. In some embodiments, the invention provides dosage forms comprising zonisamide and an antioxidant or spin trapping agent. In particular embodiments, the invention provides dosage forms comprising zonisamide in combination with allopurinol. In other particular embodiments, the invention provides dosage forms comprising zonisamide in combination with glutathione. In still further particular embodiments, the invention provides dosage forms comprising zonisamide in combination with methionine. In yet further embodiments, the invention provides dosage forms comprising zonisamide in combination with L-carnitine. In yet further embodiments, the invention provides dosage forms comprising zonisamide in combination with two or more antioxidants, such as allopurinol, glutathione, methionine, or L-carnitine. In still further embodiments, the invention provides dosage forms comprising zonisamide in combination with one or more antioxidants, such as allopurinol, glutathione, methionine, or L-carnitine, and one or more other active pharmaceutical ingredients, such as one or more NMDA antagonists, one or more SSRIs or one or more compounds having both SSRI and NMDA antagonist activity.

2. Zonisamide and NMDA Antagonist

In some embodiments, zonisamide is combined with one or more active pharmaceutical agent that block the excitotoxic actions of glutamate within the inner ear. Glutamate is a mediator of noise-induced damage to the hair cells of the inner ear and blocking N-methyl-D-aspartate (NMDA) receptors provides protection against the toxic effects of glutamate. In some embodiments, the invention provides dosage forms comprising zonisamide and a NMDA antagonist. In particular embodiments, the invention provides dosage forms comprising zonisamide in combination with riluzole. In other particular embodiments, the invention provides dosage forms comprising zonisamide in combination with caroverine. In still further particular embodiments, the invention provides dosage forms comprising zonisamide in combination with memantine. In still further embodiments, the invention provides dosage forms comprising zonisamide in combination with magnesium. In yet further embodiments, the invention provides dosage forms comprising zonisamide in combination with two or more NMDA antagonists, such as riluzole, caroverine, or memantine. In still further embodiments, the invention provides dosage forms comprising zonisamide in combination with one or more NMDA antagonists, such as riluzole, caroverine, or memantine, and one or more other active pharmaceutical ingredients, such as one or more antioxidants or spin trapping agents, one or more SSRIs or one or more compounds having both SSRI and NMDA antagonist activity.

3. Zonisamide and SSRI/NMDA Antagonist

In some embodiments, zonisamide is combined with at one or more active pharmaceutical agents that enhance synaptic levels of serotonin in the brain and enhance hearing by improving auditory processing, increasing the signal: noise ratio of environmental sounds, and/or by heightening attention. In some embodiments, the invention provides dosage forms comprising zonisamide and a selective serotonin reuptake inhibitor (SSRI). In particular embodiments, the invention provides dosage forms comprising zonisamide in combination with fluoxetine. In other particular embodiments, the invention provides dosage forms comprising zonisamide in combination with sertraline. In still further particular embodiments, the invention provides dosage forms comprising zonisamide in combination with S-citalopram.

In some advantageous embodiments, the invention provides zonisamide in combination with at least one SSRI and at least one NMDA antagonist. In exemplary embodiments, the invention provides a dosage form comprising zonisamide, at least one SSRI (such as fluoxetine, sertraline, S-citalopram or combinations of two or more thereof) and one or more members of the group of riluzole, caroverine, memantine and magnesium. In some particular embodiments, the invention provides a dosage form comprising zonisamide, at least one SSRI (such as fluoxetine, sertraline, S-citalopram or combinations of two or more thereof) and riluzole. In other particular embodiments, the invention provides a dosage form comprising zonisamide, at least one SSRI (such as fluoxetine, sertraline, S-citalopram or combinations of two or more thereof) and caroverine. In yet other embodiments, the invention provides a dosage form comprising zonisamide, at least one SSRI (such as fluoxetine, sertraline, S-citalopram or combinations of two or more thereof) and memantine. In yet other embodiments, the invention provides a dosage form comprising zonisamide, at least one SSRI (such as fluoxetine, sertraline, S-citalopram or combinations of two or more thereof) and magnesium.

In yet further embodiments, the invention provides dosage forms comprising zonisamide in combination with at least one agent having combined SSRI and NMDA antagonist activity.

In some embodiments, the invention provides dosage forms comprising zonisamide in combination with at least one SSRI agent, at least one NMDA antagonist and at least one antioxidant or spin trapping agent.

In some other embodiments, the invention provides dosage forms comprising zonisamide in combination with at least one agent having both SSRI and NMDA antagonist activity and at least one antioxidant or spin trapping agent.

4. Amantadine and Zonisamide, SSRI or an Antioxidant

The invention provides compositions comprising amantadine in combination with one or more members of the group consisting of zonisamide, selective serotonin reuptake inhibitors and antioxidants. In some embodiments, the invention provides mixtures of amantadine and one or more members of the group consisting of zonisamide, selective serotonin reuptake inhibitors and antioxidants. In other embodiments, the invention provides dosage forms combining amantadine, one or more members of the group consisting of zonisamide, selective serotonin reuptake inhibitors and antioxidants, and one or more additional excipients.

In some embodiments, the invention provides a combination of amantadine and zonisamide as the only active pharmaceutical ingredients. In other embodiments, the invention provides combinations of amantadine and one selective serotonin reuptake inhibitor as the active pharmaceutical ingredients. In other embodiments, the invention provides amantadine and one antioxidant as the active pharmaceutical ingredients. In still further embodiments, the invention provides a combination of zonisamide, a selective serotonin reuptake inhibitor and an antioxidant as the active pharmaceutical ingredients.

In some embodiments, amantadine is combined with one or more active pharmaceutical agents that bind to or metabolize reactive oxygen species and provide protection against the damage induced by oxygen species, which are toxic mediators. In some embodiments, the invention provides dosage forms comprising amantadine and an antioxidant or spin trapping agent. In particular embodiments, the invention provides dosage forms comprising amantadine in combination with allopurinol. In other particular embodiments, the invention provides dosage forms comprising amantadine in combination with glutathione. In still further particular embodiments, the invention provides dosage forms comprising amantadine in combination with methionine. In yet further embodiments, the invention provides dosage forms comprising amantadine in combination with L-carnitine. In yet further embodiments, the invention provides dosage forms comprising amantadine in combination with two or more antioxidants, such as allopurinol, glutathione, methionine, or L-carnitine.

In some embodiments, amantadine is combined with one or more selective serotonin reuptake inhibitors, such as fluoxetine, sertraline or S-citolopram. In particular embodiments, the invention provides dosage forms comprising amantadine in combination with fluoxetine. In other particular embodiments, the invention provides dosage forms comprising amantadine in combination with sertraline. In still further particular embodiments, the invention provides dosage forms comprising amantadine in combination with S-citalopram. In yet further embodiments, the invention provides dosage forms comprising amantadine in combination with two or more members of the group consisting of fluoxetine, sertraline and S-citalopram.

In some embodiments, amantadine is combined with pirlindole.

5. Pirlindole Plus NMDA Antagonist

In some embodiments, the invention provides combinations of pirlindole and a N-methyl-D-aspartate antagonist. In particular embodiments, the invention provides dosage forms comprising pirlindole in combination with riluzole. In other particular embodiments, the invention provides dosage forms comprising pirlindole in combination with caroverine. In still further particular embodiments, the invention provides dosage forms comprising pirlindole in combination with memantine. In yet further embodiments, the invention provides dosage forms comprising pirlindole in combination with two or more NMDA antagonists, such as riluzole, caroverine, or memantine. In yet further embodiments, the invention provides dosage forms comprising pirlindole in combination with two or more NMDA antagonists, such as riluzole, caroverine, or magnesium.

6. Calcium Channel Antagonists and SSRIs, NSRIs

In some embodiments, the invention provides at least one calcium channel antagonist, such as nimodipine or verapamil, in combination with one or more SSRIs. In other embodiments, the invention provides at least one calcium channel antagonist, such as nimodipine or verapamil, in combination with one or more NSRIs. In still further embodiments, the invention provides at least one calcium channel antagonist, such as nimodipine or verapamil, in combination with one or more SSRIs and at one or more NSRIs.

In some embodiments, the invention provides at least one calcium channel antagonist, such as nimodipine or verapamil, in combination with one or more selective serotonin reuptake inhibitors selected from the group consisting of fluoxetine, sertraline and S-citalopram. In some particular embodiments, the invention provides nimodipine in combination with one or more selective serotonin reuptake inhibitors. In other particular embodiments, the invention provide verapamil in combination with one or more selective serotonin reuptake inhibitors. In other particular embodiments, the invention provides combinations of nimodipine, verapamil and one or more selective serotonin reuptake inhibitors.

In other embodiments, the invention provides at least one calcium channel antagonist, such as nimodipine or verapamil, in combination with one or more norepinephrine-serotonin reuptake inhibitors. In some particular embodiments, the invention provides nimodipine in combination with one or more norepinephrine-serotonin reuptake inhibitors. In other particular embodiments, the invention provide verapamil in combination with one or more norepinephrine-serotonin reuptake inhibitors. In other particular embodiments, the invention provides combinations of nimodipine, verapamil and one or more norepinephrine-serotonin reuptake inhibitors.

In other embodiments, the invention provides at least one calcium channel antagonist, such as nimodipine or verapamil, in combination with one or more selective serotonin reuptake inhibitors and one or more norepinephrine-serotonin reuptake inhibitors.

7. Indeloxazine Plus NMDA Antagonist

In some embodiments, the invention provides combinations of indeloxazine and a N-methyl-D-aspartate antagonist, such as riluzole, caroverine, memantine, magnesium or combinations thereof. In particular embodiments, the invention provides dosage forms comprising indeloxazine in combination with riluzole. In other particular embodiments, the invention provides dosage forms comprising indeloxazine in combination with caroverine. In still further particular embodiments, the invention provides dosage forms comprising indeloxazine in combination with memantine. In still further particular embodiments, the invention provides dosage forms comprising indeloxazine in combination with magnesium. In yet further embodiments, the invention provides dosage forms comprising indeloxazine in combination with two or more NMDA antagonists, such as riluzole, carovenne, memantine or magnesium.

III. Methods of Use

A. General Administration Protocol p The zonisamide compositions are administered in an effective dosage to protect the mammal from hearing loss, to restore lost hearing or to alleviate one or more symptoms of a hearing disorder, such as decreased speech recognition, tinnitus, vertigo or decreased memory. In some embodiments, zonisamide is administered as the sole active pharmaceutical ingredient. In other embodiments, zonisamide is combined with one or more additional active pharmaceutical ingredients. In this context “combined” means using zonisamide and at least one additional active pharmaceutical ingredient in the same regime. Thus, zonisamide and at least one other active pharmaceutical ingredient can be combined in the same dosage form and thereby administered simultaneously to the mammal being treated. Alternatively, zonisamide and at least one other active pharmaceutical ingredient can be compounded (prepared) in separate dosage forms and administered separately, either at substantially the same time (simultaneously) or at substantially different times (sequentially) in a given period of time. For example, zonisamide may be administered at bedtime and the other active pharmaceutical ingredient may be administered upon awakening. As another example, zonisamide and another active pharmaceutical ingredient may be taken on alternating days. As a further example, zonisamide and another active pharmaceutical ingredient may be taken at substantially the same time. In specific cases, at least one additional active pharmaceutical ingredient may be taken at such a time relative to the dosing of zonisamide that it counteracts one or more unwanted side effects of zonisamide.

In some embodiments, the compositions are administered orally, although other dosing regimens are possible and are not excluded from the invention. In one embodiment, the zonisamide and a second active pharmaceutical ingredient are administered simultaneously, either in the same or separate dosage forms. The compositions can be administered as immediate release, sustained release, intermittent release, and/or delayed release formulations. The composition can be administered in a single dose, an escalating dose, or administered at an elevated dosage which is then decreased to a lower dosage after a particular circulating blood concentration of the compound has been achieved. In some embodiments, the drugs are administered in an immediate release twice/day dosing regimen in which the second active pharmaceutical ingredient is given twice daily (BID) in the normal dose range and zonisamide is given twice daily (BID) at half its daily dose.

An intermittent administration protocol may be used where chronic administration is not desirable. The compound or formulation is administered in time blocks of several days with a defmed minimum washout time between blocks. Intermittent administration occurs over a period of several weeks to months to achieve a significant improvement in the symptoms of sleep-related breathing disorders.

One of skill in the art would be able to choose administration protocols and determine appropriate dosing regimes to treat symptoms of sleep-related breathing disorders based on bioavailability and half-life of the compound to be administered. For many of the disclosed compounds, appropriate dosage ranges have been established to maximize circulating concentrations of the compound and minimize side-effects.

The combination of zonisamide and antioxidant, NMDA antagonist, SSRI or combined SSRI/NMDA antagonist combination can be administered for a specific duration to improve symptoms of a particular disorder. A suitable endpoint can be where one symptom of the disorder is treated by administration of the compounds and the treatment considered effective. In other situations, the treatment can be considered effective when more than one symptom is treated.

B. Effective Dosage Ranges

Appropriate dosages can be determined by one of skill in the art based on using routine experimentation and standard techniques utilizing dosages currently approved. Compounds in the disclosed drug classes are known in the art and can be initially administered at similar doses and titrated appropriately to treat symptoms of the disorders and side effects in a given patient. Intra-patient variability is known in the art depending on the severity of symptoms and dosages are commonly adjusted to exact a particular therapeutic effect in a particular patient.

Therapeutically effective amounts for use in humans can also be determined from animal models. For example, a dose for humans can be formulated to achieve a circulating concentration that has been found to be effective in animals. Effective amounts for use in humans can also be determined from human data for the compounds used to treat other disorders, for example, neurological disorders. The amount administered can be the same amount administered to treat other neurological disorders or can be an amount higher or lower than the amount administered to treat other neurological disorders.

The optimal concentration of the drug in each pharmaceutical formulation varies according to the formulation itself. Typically, the pharmaceutical formulation contains the active pharmaceutical ingredient (API) at a concentration of about 0.1 to 90% by weight (such as about 1-20% or 1-10%). Appropriate dosages of the API can readily be determined by those of ordinary skill in the art of medicine by assessing amelioration of the disorder or side effect in the patient, and increasing the dosage and/or frequency of treatment as desired. The optimal amount of the API may depend upon the mode of administration, the age and the body weight of the patient, and the condition of the patient. Typically, the API is administered at a dosage of 0.1 to 1.0 mg/kg. Preferred daily doses of zonisamide are approximately 50 to 600 mg/day, and preferably 100 to 400 mg/day. Preferred daily doses of the antioxidants, NMDA antagonists, SSRI or combined SSRI/NMDA antagonists are approximately 1 to 500 mg/day, and preferably 4 to 250 mg/day.

C. Conditions to be Treated

The invention provides methods of treating or preventing various hearing disorders. The hearing disorders to be treated or prevented include noise-induced hearing loss, drug-induced hearing loss, central auditory hearing disorder (CAPD), tinnitus and presbyacusis.

1. Noise-Induced Hearing Loss

In particular embodiments, the invention provides methods of protecting against noise-induced damage or loss of hair cells in the inner cochlea of the inner ear. The method comprises administering a therapeutic composition of the invention alone or in combination with one or more additional active ingredients to a mammal in need of protection from noise-induced hearing loss in an amount sufficient to protect against noise-induced hearing loss. In some embodiments, a therapeutic composition of the invention is combined with at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof, in the same dosage form. In other embodiments, a therapeutic composition of the invention is administered to the mammal in a dosage form separate from at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof.

In some embodiments, therapeutic compositions for treatment or prevention of noise-induced hearing loss according to the invention comprise one or more compounds selected from the group consisting of: antioxidants and/or spin-trapping agents; N-methyl-D-aspartate (NMDA) antagonists; selective serotonin reuptake inhibitor (SSRI)/NMDA antagonists; dopamine releaser/NMDA antagonists; acetylcholine release inducer/antioxidant/NMDA antagonist/norepinephrine-epinephrine reuptake inhibitors; monamineoxidase-A/serotonin reuptake inhibiting/antioxidants; norepinephrine and serotonin reuptake inhibitor/low-affinity NMDA antagonists; calcium channel antagonists and SSRI or norepinephrine selective reuptake inhibitor (NSRI); 5HT serotonin reuptake inhibiting, norepinephrine reuptake inhibiting, acetylcholine releasing and N-methyl-D-aspartate antagonists.

In some particular embodiments therapeutic compositions for treatment or prevention of noise-induced hearing loss according to the invention comprise one or more antioxidants and/or spin-trapping agents selected from the group consisting of: allopurinol, glutathione, methionine, L-carnitine, and combinations, pharmaceutically acceptable salts and polymorphs thereof.

In other particular embodiments, therapeutic compositions for treatment or prevention of noise-induced hearing loss according to the invention comprise one or more N-methyl-D-aspartate (NMDA) antagonists selected from the group consisting of: riluzole, caroverine, memantine, magnesium and combinations, pharmaceutically acceptable salts and polymorphs thereof.

In further particular embodiments, therapeutic compositions for treatment or prevention of noise-induced hearing loss according to the invention comprise one or more selective serotonin reuptake inhibitor (SSRI)/NMDA antagonists selected from the group consisting of: alaproclate and combinations of one or more SSRIs with one or more NMDA antagonists. Suitable SSRIs include fluoxetine, sertraline, S-citalopram and combinations thereof. Suitable NMDA antagonists include riluzole, caroverine, memantine and combinations, pharmaceutically acceptable salts and polymorphs thereof.

In still further particular embodiments therapeutic compositions for treatment or prevention of noise-induced hearing loss according to the invention comprise one or more dopamine releaser/NMDA antagonists selected from the group consisting of: amantadine and one or more combinations of compounds selected from: amantadine and zonisamide; amantadine and a SSRI; amantadine and an antioxidant; amantadine, zonisamide and a SSRI; amantadine, zonisamide and an antioxidant; amantadine, a SSRI and an antioxidant; and amantadine, zonisarnide, a SSRI and an antioxidant, and pharmaceutically acceptable salts, polymorphs and combinations thereof.

In particular embodiments, therapeutic compositions for treatment or prevention of noise-induced hearing loss according to the invention comprise one or more acetylcholine release inducer/antioxidant/NMDA antagonist/norepinephrine-epinephrine reuptake inhibitors selected from the group consisting of: bifemelane, and pharmaceutically acceptable salts and/or polymorphs thereof.

In particular embodiments, therapeutic compositions for treatment or prevention of noise-induced hearing loss according to the invention comprise one or more monamineoxidase-A/serotonin reuptake inhibiting/antioxidants selected from the group consisting of: pirlindole, and pharmaceutically acceptable salts and/or polymorphs thereof.

In particular embodiments, therapeutic compositions for treatment or prevention of noise-induced hearing loss according to the invention comprise one or more norepinephrine and serotonin reuptake inhibitor/low-affinity NMDA antagonists selected from the group consisting of: milnacipran, bicifadine, and pharmaceutically acceptable salts, polymorphs and combinations thereof.

In particular embodiments, therapeutic compositions for treatment or prevention of noise-induced hearing loss according to the invention comprise one or more calcium channel antagonists and SSRI or norepinephrine selective reuptake inhibitors (NSRIs); selected from the group consisting of: nimodipine, verapamil and pharmaceutically acceptable salts, polymorphs and combinations thereof.

In particular embodiments, therapeutic compositions for treatment or prevention of noise-induced hearing loss according to the invention comprise one or more 5HT serotonin reuptake inhibiting, norepinephrine reuptake inhibiting, acetylcholine releasing and N-methyl-D-aspartate antagonists; selected from the group consisting of: indeloxazine, a combination of indeloxazine with at least one NMDA antagonist, such as riluzole, caroverine, memantine, magnesium, and pharmaceutically acceptable salts, polymorphs and combinations thereof.

2. Drug-Induced Hearing Loss

In some embodiments, the invention provides methods of treating or preventing one or more drug-induced hearing disorders. The method comprises administering to a patient a therapeutically effective amount of a composition of the invention alone or in combination with one or more additional active ingredient. In some embodiments, the composition of the invention further comprises at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof, in the same dosage form. In other embodiments, the composition of the invention is administered to the patient in a dosage form separate from at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof.

In some embodiments, the invention provides a method for preventing a drug-induced hearing disorder, such as hearing loss or tinnitus. In some such embodiments, the invention provides a method for preventing the onset of a drug-induced hearing disorder. In particular, the invention provides methods for preventing a drug-induced hearing disorder, comprising administering a therapeutically effective amount of composition of the invention to a patient prior to administering to that patient one or more chemotherapeutic agents for the treatment of a condition other than a hearing disorder. In some embodiments, treatment with the composition comprising of the invention can begin substantially before treatment with the other chemotherapeutic agent. (For purposes of this invention, “other chemotherapeutic agent” includes antineoplastic and antibacterial compounds as described in more detail below.) In exemplary embodiments, treatment with a therapeutic composition of the invention can begin up to one month prior to treatment with the other chemotherapeutic agent, although prophylactic pre-treatment can vary from about 1 day to about 60 days, depending upon the particular chemotherapy contemplated.

In still further embodiments, the invention provides a method of preventing ototoxic effects of one or more chemotherapeutic agents, which includes concurrent dosing of a therapeutic composition of the invention with the other chemotherapeutic agent. (Such ototoxic effects include drug-induced hearing loss and tinnitus.) In some embodiments, dosing of the therapeutic composition of the invention begins on the same day as dosing of the other chemotherapeutic agent. Treatment with a composition of the invention can then be continued for the duration of chemotherapeutic agent, or may continue for some time, e.g. from about 1 to about 90 days after cessation of the chemotherapeutic agent. In some cases, it will be sufficient to continue administration of the therapeutic composition of the invention for a time after cessation of administration of the other chemotherapeutic agent. In some embodiments, such period of time is equivalent to the washout period (plus or minus a few days) for the other chemotherapeutic agent. The washout period will vary based on the rate of clearance of the chemotherapeutic agent from the body, and can be determined by one of skill in the art by art-recognized methods.

In other embodiments, the invention provides a method for treating a drug-induced hearing disorder, such as drug-induced hearing loss or tinnitus. Such treatment can include amelioration of drug-induced hearing loss, reduction or elimination of tinnitus, partial or total rehabilitation of hearing, or prevention of further hearing loss arising out of ototoxic effects of chemotherapeutic agents. In such embodiments, the invention provides for dosing of a therapeutic composition of the invention in response to a noted decrease in hearing function arising out of, or occurring during, dosing of one or more chemotherapeutic agents. Dosing of a therapeutic composition of the invention can then continue for the duration of chemotherapeutic treatment, and for at least one day after cessation of chemotherapeutic treatment. In some embodiments, dosing of the therapeutic composition of the invention can continue for at least a washout period for the chemotherapeutic agent after the last dose of chemotherapeutic agent has be administered to the patient. In particular embodiments, dosing of the therapeutic composition of the invention can persist for about 1 to 90 days after cessation of chemotherapy. In other embodiments, dosing of the therapeutic composition of the invention can persist indefinitely, for example until the clinician is satisfied that the danger of ototoxic hearing loss has passed or until the clinician determines that drug-induced hearing loss and/or tinnitus has been ameliorated to a desired degree.

A number of drugs have been found to elicit ototoxic effects in at least some patients. Embodiments of the invention comprise treatment of ototoxic sided effects, such as hearing loss and tinnitus, in a patient has been, is being or will be treated with one or more ototoxic drugs. Some examples of ototoxic drugs include certain antibacterial and antineoplastic drugs. For example, some ototoxic drugs contemplated within the scope of the present invention are chemotherapeutic agents, e.g. antineoplastic agents, and antibiotics. Other possible candidates include loop-diuretics, quinines or a quinine-like compound, and salicylate or salicylate-like compounds. Thus, the present invention provides a method for treating or preventing a drug-induced hearing disorder caused by an ototoxic agent, wherein the ototoxic agent is an antineoplastic agent such as cisplatin, an antibiotic such as an aminoglycoside, a loop-diuretic, a quinine, a quinine-like compound, a salicylate or salicylate-like compound. The method comprises administering to a patient a therapeutically effective amount of a therapeutic composition of the invention, wherein the therapeutically effective amount is an amount sufficient to treat or prevent a drug-induced hearing disorder, such as drug-induced hearing loss and/or tinnitus.

In particular embodiments, the invention provides methods for treating or preventing a drug-induced hearing disorder, such as drug-induced hearing loss or tinnitus, wherein the ototoxic agent is an antineoplastic agent such as cisplatin, an antibiotic such as an aminoglycoside (as described below), a loop-diuretic, a quinine, a quinine-like compound, a salicylate or salicylate-like compound. The method comprises administering to a patient a therapeutically effective amount of a therapeutic composition of the invention. The drug-induced hearing disorder may include drug-induced hearing loss, tinnitus or both.

Ototoxic aminoglycoside antibiotics include but are not limited to neomycin, paromomycin, ribostamycin, lividomycin, kanamycin, amikacin, tobramycin, viomycin, gentamicin, sisomicin, netilmicin, streptomycin, dibekacin, fortimicin, and dihydrostreptomycin, or combinations thereof. Particular antibiotics include neomycin B, kanamycin A, kanamycin B, gentamicin C1, gentamicin C1a, and gentamicin C2. Thus, embodiments of the present invention provide methods of treating or preventing a drug-induced hearing disorder, such as drug-induced hearing loss or tinnitus, comprising administering to a patient, who has been, is being or will be treated with one or more aminoglycosides, a therapeutically effective amount of a composition of the invention.

Hearing impairments induced by aminoglycosides can be prevented or reduced by the methods of the invention. Although the aminoglycosides are particularly useful due to their rapid bactericidal action against infections of aminoglycoside-susceptible organisms, their use has heretofore been limited to more severe, complicated infections because of ototoxic and nephrotoxic side-effects. For this reason the aminoglycosides have been considered to have a low therapeutic/risk ratio compared to other antibiotics used systemically. Thus, the invention provides improved methods of treatment of aminoglycoside-susceptible infections, comprising administering to a patient an ant bacterially effective amount of an aminoglycoside and a therapeutic composition of the invention. It is to be recognized that therapeutic doses of aminoglycosides have been established; and the present invention contemplates administration of aminoglycosides in a range of about 100 to about 500%, in particular about 100 to about 250%, and more particularly about 100 to about 150% of the currently recommended doses, which are available in general in the product labeling and package inserts for the commercially available drug aminoglycoside drug products. The improved methods provide prophylaxis against aminoglycoside-induced hearing loss and/or tinnitus, thereby expanding the therapeutic index of the aminoglycoside drug.

In some embodiments the composition of the invention is co-administered with an ototoxin in the same dosage form. For example, an improved method is provided for treatment of infection of a mammal by administration of an aminoglycoside antibiotic, the improvement comprising administering a therapeutically effective amount of a composition of the invention and an antibiotic.

In other embodiments, the aminoglycoside antibiotic and the therapeutic composition of the invention are administered to the patient in separate dosage forms.

In yet another embodiment is provided an improved method for treatment of cancer in a mammal by administration of a chemotherapeutic compound; the improvement comprising administering to a patient a therapeutically effective amount of a composition of the invention and an antineoplastic agent.

In other embodiments, the antineoplastic agent and the therapeutic composition of the invention are administered to the patient in separate dosage forms.

Ototoxic antineoplastic chemotherapeutic agents include cisplatin or cisplatin-like compounds, taxol or taxol-like compounds, and other chemotherapeutic agents believed to cause ototoxin-induced hearing impairments, e.g., vincristine, an antineoplastic drug used to treat hematological malignancies and sarcomas. Thus, the methods of the invention may be used to treat ototoxicity, (such as drug-induced hearing loss), in a patient that will be, is being, or has been treated with an antineoplastic agent, including cisplatin or cisplatin-like compounds, taxol or taxol-like compounds, and other chemotherapeutic agents believed to cause ototoxin-induced hearing impairments, e.g., vincristine, an antineoplastic drug used to treat hematological malignancies and sarcomas.

In some embodiments, therapeutic compositions for treatment of drug-induced hearing loss according to the invention comprise one or more compounds selected from the group consisting of: antioxidants and/or spin-trapping agents; N-methyl-D-aspartate (NMDA) antagonists; selective serotonin reuptake inhibitor (SSRI)/NMDA antagonists; dopamine releaser/NMDA antagonists; acetylcholine release inducer/antioxidant/NMDA antagonist/norepinephrine-epinephrine reuptake inhibitors; monamineoxidase-A/serotonin reuptake inhibiting/antioxidants; norepinephrine and serotonin reuptake inhibitor/low-affinity NMDA antagonists; calcium channel antagonists and SSRI or norepinephrine selective reuptake inhibitor (NSRI); 5HT serotonin reuptake inhibiting, norepinephrine reuptake inhibiting, acetylcholine releasing and N-methyl-D-aspartate antagonists.

In some particular embodiments, therapeutic compositions for treatment of drug-induced hearing loss according to the invention comprise one or more antioxidants and/or spin-trapping agents selected from the group consisting of: allopurinol, glutathione, methionine, L-carnitine, and combinations, pharmaceutically. acceptable salts and polymorphs thereof.

In other particular embodiments, therapeutic compositions for treatment of drug-induced hearing loss according to the invention comprise one or more N-methyl-D-aspartate (NMDA) antagonists selected from the group consisting of: riluzole, caroverine, memantine, magnesium and combinations, pharmaceutically acceptable salts and polymorphs thereof.

In further particular embodiments, therapeutic compositions for treatment of drug-induced hearing loss according to the invention comprise one or more selective serotonin reuptake inhibitor (SSRI)/NMDA antagonists selected from the group consisting of: alaproclate and combinations of one or more SSRIs with one or more NMDA antagonists. Suitable SSRIs include fluoxetine, sertraline, S-citalopram and combinations thereof. Suitable NMDA antagonists include riluzole, caroverine, memantine and combinations, pharmaceutically acceptable salts and polymorphs thereof.

In still further particular embodiments, therapeutic compositions for treatment of drug-induced hearing loss according to the invention comprise one or more dopamine releaser/NMDA antagonists selected from the group consisting of: amantadine and one or more combinations of compounds selected from: amantadine and zonisamide; amantadine and a SSRI; amantadine and an antioxidant; amantadine, zonisamide and a SSRI; amantadine, zonisamide and an antioxidant; amantadine, a SSRI and an antioxidant; and amantadine, zonisamide, a SSRI and an antioxidant, and pharmaceutically acceptable salts, polymorphs and combinations thereof.

In particular embodiments, therapeutic compositions for treatment of drug-induced hearing loss according to the invention comprise one or more acetylcholine release inducer/antioxidant/NMDA antagonist/norepinephrine-epinephrine reuptake inhibitors selected from the group consisting of: bifemelane, and pharmaceutically acceptable salts and/or polymorphs thereof.

In particular embodiments, therapeutic compositions for treatment of drug-induced hearing loss according to the invention comprise one or more monamineoxidase-A/serotonin reuptake inhibiting/antioxidants selected from the group consisting of: pirlindole, and pharmaceutically acceptable salts and/or polymorphs thereof.

In particular embodiments, therapeutic compositions for treatment of drug-induced hearing loss according to the invention comprise one or more norepinephrine and serotonin reuptake inhibitor/low-affinity NMDA antagonists selected from the group consisting of: milnacipran, bicifadine, and pharmaceutically acceptable salts, polymorphs and combinations thereof.

In particular embodiments, therapeutic compositions for treatment of drug-induced hearing loss according to the invention comprise one or more calcium channel antagonists and SSRI or norepinephrine selective reuptake inhibitors (NSRIs); selected from the group consisting of: nimodipine, verapamil and pharmaceutically acceptable salts, polymorphs and combinations thereof.

In particular embodiments, therapeutic compositions for treatment of drug-induced hearing loss according to the invention comprise one or more 5HT serotonin reuptake inhibiting, norepinephrine reuptake inhibiting, acetylcholine releasing and N-methyl-D-aspartate antagonists; selected from the group consisting of: indeloxazine, a combination of indeloxazine with at least one NMDA antagonist, such as riluzole, caroverine, memantine, magnesium, and pharmaceutically acceptable salts, polymorphs and combinations thereof.

3. Central Auditory Hearing Disorder (CAPD)

Central auditory processing (CAP) relates to the efficiency and efficacy with which the central nervous system (CNS) utilizes auditory information. More specifically, CAP relates to the perceptual processing of auditory information in the CNS as well as the neurobiological activity that underlies that processing and gives rise to electro-physiological auditory potentials. CAP includes the auditory mechanisms that underlie the following abilities or skills: sound localization and lateralization; auditory discrimination; auditory pattern recognition; temporal aspects of audition, including temporal integration, temporal discrimination (e.g. temporal gap detection), temporal ordering and temporal masking; auditory performance in competing acoustic signals (including dichotic listening) and auditory performance with degraded acoustic signals. Central auditory processing disorders, then, relate to difficulties in the perceptual processing of auditory information in the CNS as demonstrated by poor performance in one or more of the above skills. Central auditory processing disorders (CAPD). In some embodiments of the invention, there are provided methods for treatment of CAPD, comprising administering to a patient a therapeutically effective amount of a therapeutic composition according to the invention.

Tests for CAPD are known in the art. See generally, American Speech-Language Hearing Association, (Central) Auditory Processing Disorders, 1-20, (2005), available at http://www.asha.org/members/deskref-joumals/deskref/default. Suitable tests available for central auditory assessment include: auditory discrimination tests; auditory temporal processing and patterning tests; dichotic speech tests; monaural low-redundancy speech tests; binaural interaction tests; electroacoustic measures; and electrophysiologic measures. Such methods are known to known audiologists and are described generally in American Speech-Language Hearing Association, (Central) Auditory Processing Disorders, 6-7.

In some embodiments, therapeutic compositions for the treatment or prevention of CAPD according to the invention comprise one or more compounds selected from the group consisting of: antioxidants and/or spin-trapping agents; N-methyl-D-aspartate (NMDA) antagonists; selective serotonin reuptake inhibitor (SSRI)/NMDA antagonists; dopamine releaser/NMDA antagonists; acetylcholine release inducer/antioxidant/NMDA antagonist/norepinephrine-epinephrine reuptake inhibitors; monamineoxidase-A/serotonin reuptake inhibiting/antioxidants; norepinephrine and serotonin reuptake inhibitor/low-affinity NMDA antagonists; calcium channel antagonists and SSRI or norepinephrine selective reuptake inhibitor (NSRI); SHT serotonin reuptake inhibiting, norepinephrine reuptake inhibiting, acetylcholine releasing and N-methyl-D-aspartate antagonists.

In some particular embodiments, therapeutic compositions for the treatment or prevention of CAPD according to the invention comprise one or more antioxidants and/or spin-trapping agents selected from the group consisting of: allopurinol, glutathione, methionine, L-carnitine, and combinations, pharmaceutically acceptable salts and polymorphs thereof.

In other particular embodiments, therapeutic compositions for the treatment or prevention of CAPD according to the invention comprise one or more N-methyl-D-aspartate (NMDA) antagonists selected from the group consisting of: riluzole, caroverine, memantine, magnesium and combinations, pharmaceutically acceptable salts and polymorphs thereof.

In further particular embodiments, therapeutic compositions for the treatment or prevention of CAPD compositions according to the invention comprise one or more selective serotonin reuptake inhibitor (SSRI)/NMDA antagonists selected from the group consisting of: alaproclate and combinations of one or more SSRIs with one or more NMDA antagonists. Suitable SSRIs include fluoxetine, sertraline, S-citalopram and combinations thereof. Suitable NMDA antagonists include riluzole, caroverine, memantine and combinations, pharmaceutically acceptable salts and polymorphs thereof.

In still further particular embodiments, therapeutic compositions for the treatment or prevention of CAPD therapeutic compositions according to the invention comprise one or more dopamine releaser/NMDA antagonists selected from the group consisting of: amantadine and one or more combinations of compounds selected from: amantadine and zonisamide; amantadine and a SSRI; amantadine and an antioxidant; amantadine, zonisamide and a SSRI; amantadine, zonisamide and an antioxidant; amantadine, a SSRI and an antioxidant; and amantadine, zonisamide, a SSRI and an antioxidant, and pharmaceutically acceptable salts, polymorphs and combinations thereof.

In particular embodiments, therapeutic compositions for the treatment or prevention of CAPD therapeutic compositions according to the invention comprise one or more acetylcholine release inducer/antioxidant/NMDA antagonist/norepinephrine-epinephrine reuptake inhibitors selected from the group consisting of: bifemelane, and pharmaceutically acceptable salts and/or polymorphs thereof.

In particular embodiments, therapeutic compositions for the treatment or prevention of CAPD according to the invention comprise one or more monamineoxidase-A/serotonin reuptake inhibiting/antioxidants selected from the group consisting of: pirlindole, and pharmaceutically acceptable salts and/or polymorphs thereof.

In particular embodiments, therapeutic compositions for the treatment or prevention of CAPD according to the invention comprise one or more norepinephrine and serotonin reuptake inhibitor/low-affinity NMDA antagonists selected from the group consisting of: milnacipran, bicifadine, and pharmaceutically acceptable salts, polymorphs and combinations thereof.

In particular embodiments, therapeutic compositions for the treatment or prevention of CAPD according to the invention comprise one or more calcium channel antagonists and SSRI or norepinephrine selective reuptake inhibitors (NSRIs); selected from the group consisting of: nimodipine, verapamil and pharmaceutically acceptable salts, polymorphs and combinations thereof.

In particular embodiments, therapeutic compositions for the treatment or prevention of CAPD according to the invention comprise one or more 5HT serotonin reuptake inhibiting, norepinephrine reuptake inhibiting, acetylcholine releasing and N-methyl-D-aspartate antagonists; selected from the group consisting of: indeloxazine, a combination of indeloxazine with at least one NMDA antagonist, such as riluzole, caroverine, memantine, magnesium, and pharmaceutically acceptable salts, polymorphs and combinations thereof.

4. Tinnitus

Tinnitus is the perception of sound in the ears even without external auditory stimulation. The most frequent manifestation of tinnitus is a ringing in the ears; however, tinnitus can also present as crickets, whooshing, pulsing, ocean waves, buzzing, even music, Tinnitus may be temporary, intermittent or even permanent; and its severity can range from a quiet background ringing to an overwhelming auditory sensation that drowns out external sources of sound

Tinnitus may be caused by one or more factors, such as administration of, or exposure to, ototoxic substances (such as an aspirin overdose), exposure to a short burst of extreme noise (e.g. gunshot or explosion) or prolonged exposure to high decibel noise (such as aircraft engine noise, high decibel music concerts or high decibel headphone usage), or central auditory processing disorders as discussed herein.

Aside from hearing loss, tinnitus is one of the most prevalent symptoms of hearing disorders, and can cause annoyance or even major disruption in the lives of those who suffer from it. Some sources estimate that there are over 50 million persons who suffer from tinnitus in the United States. Of these, an estimated 12 million will seek medical attention for tinnitus. Moreover, about two million tinnitus patients are so seriously debilitated that they cannot function on a “normal,” day-to-day basis.

Thus, embodiments of the invention provide a method of treating or preventing tinnitus. The method includes administering a therapeutically effective amount of a therapeutic composition of the invention to the patient.

In some embodiments, the invention provides a method of preventing drug-induced tinnitus. The method entails administration of a therapeutic composition prior to administration of a drug known to cause ototoxic tinnitus in a patient, as discussed in more detail above. Administration of the therapeutic composition of the invention can then continue until administration of, or exposure to, the ototoxin has ceased. In some embodiments, administration of the therapeutic composition of the invention may be continued for some period after administration of, or exposure to, the ototoxin has ceased. In some embodiments, such time period is equal to or greater than a wash-out period for the ototoxin.

In some embodiments, therapeutic compositions for the prevention or treatment of tinnitus according to the invention comprise one or more compounds selected from the group consisting of: antioxidants and/or spin-trapping agents; N-methyl-D-aspartate (NMDA) antagonists; selective serotonin reuptake inhibitor (SSRI)/NMDA antagonists; dopamine releaser/NMDA antagonists; acetylcholine release inducer/antioxidant/NMDA antagonist/norepinephrine-epinephrine reuptake inhibitors; monamineoxidase-A/serotonin reuptake inhibiting/antioxidants; norepinephrine and serotonin reuptake inhibitor/low-affinity NMDA antagonists; calcium channel antagonists and SSRI or norepinephrine selective reuptake inhibitor (NSRI); 5HT serotonin reuptake inhibiting, norepinephrine reuptake inhibiting, acetylcholine releasing and N-methyl-D-aspartate antagonists.

In some particular embodiments, therapeutic compositions for the prevention or treatment of tinnitus according to the invention comprise one or more antioxidants and/or spin-trapping agents selected from the group consisting of: allopurinol, glutathione, methionine, L-carnitine, and combinations, pharmaceutically acceptable salts and polymorphs thereof.

In other particular embodiments, therapeutic compositions for the prevention or treatment of tinnitus according to the invention comprise one or more N-methyl-D-aspartate (NMDA) antagonists selected from the group consisting of: riluzole, caroverine, memantine, magnesium and combinations, pharmaceutically acceptable salts and polymorphs thereof.

In further particular embodiments, therapeutic compositions for the prevention or treatment of tinnitus according to the invention comprise one or more selective serotonin reuptake inhibitor (SSRI)/NMDA antagonists selected from the group consisting of: alaproclate and combinations of one or more SSRIs with one or more NMDA antagonists. Suitable SSRIs include fluoxetine, sertraline, S-citalopram and combinations thereof. Suitable NMDA antagonists include riluzole, caroverine, memantine and combinations, pharmaceutically acceptable salts and polymorphs thereof.

In still further particular embodiments, therapeutic compositions for the prevention or treatment of tinnitus according to the invention comprise one or more dopamine releaser/NMDA antagonists selected from the group consisting of: amantadine and one or more combinations of compounds selected from: amantadine and zonisamide; amantadine and a SSRI; amantadine and an antioxidant; amantadine, zonisamide and a SSRI; amantadine, zonisamide and an antioxidant; amantadine, a SSRI and an antioxidant; and amantadine, zonisamide, a SSRI and an antioxidant, and pharmaceutically acceptable salts, polymorphs and combinations thereof.

In particular embodiments, therapeutic compositions for the prevention or treatment of tinnitus according to the invention comprise one or more acetylcholine release inducer/antioxidant/NMDA antagonist/norepinephrine-epinephrine reuptake inhibitors selected from the group consisting of: bifemelane, and pharmaceutically acceptable salts and/or polymorphs thereof.

In particular embodiments, therapeutic compositions for the prevention or treatment of tinnitus according to the invention comprise one or more monamineoxidase-A/serotonin reuptake inhibiting/antioxidants selected from the group consisting of: pirlindole, and pharmaceutically acceptable salts and/or polymorphs thereof.

In particular embodiments, therapeutic compositions for the prevention or treatment of tinnitus according to the invention comprise one or more norepinephrine and serotonin reuptake inhibitor/low-affinity NMDA antagonists selected from the group consisting of: milnacipran, bicifadine, and pharmaceutically acceptable salts, polymorphs and combinations thereof.

In particular embodiments therapeutic compositions for the prevention or treatment of tinnitus according to the invention comprise one or more calcium channel antagonists and SSRI or norepinephrine selective reuptake inhibitors (NSRIs); selected from the group consisting of: nimodipine, verapamil and pharmaceutically acceptable salts, polymorphs and combinations thereof.

In particular embodiments, therapeutic compositions for the prevention or treatment of tinnitus according to the invention comprise one or more 5HT serotonin reuptake inhibiting, norepinephrine reuptake inhibiting, acetylcholine releasing and N-methyl-D-aspartate antagonists; selected from the group consisting of: indeloxazine, a combination of indeloxazine with at least one NMDA antagonist, such as riluzole, caroverine, memantine, magnesium, and pharmaceutically acceptable salts, polymorphs and combinations thereof.

5. Presbyacusis

Presbycusis (or Presbyacusis) is age-related hearing loss, which is the most common form of hearing loss in persons over 55 years of age. Age-related hearing loss develops gradually over time and in its early stages may be practically imperceptible to the affected individual. The cause of age-related hearing loss is generally considered to be degeneration of the auditory nervous system, especially the auditory nerves in the ears.

Embodiments of the invention provide methods of treating or preventing presbycusis, comprising administering to a patient a therapeutically effective amount of a therapeutic composition according to the invention. In some embodiments, the invention provides a method of preventing age-related degradation of hearing, comprising administering to a patient a therapeutically effective amount of a therapeutic composition of the invention. In particular embodiments, the therapeutically effective amount of the therapeutic composition is an amount sufficient to reduce or eliminate the loss of hearing due to aging of the auditory nervous system.

Embodiments of the invention also provide methods of treating presbycusis. In some embodiments, the invention provides a method of treating age-related degradation of hearing, comprising administering to a patient a therapeutically effective amount of a therapeutic composition of the invention. In particular embodiments, the method entails administering to a person suffering from age-related hearing loss a therapeutically effective amount of a therapeutic composition of the invention. In such embodiments, the therapeutically effective amount of the therapeutic composition of the invention is that amount of therapeutic composition effective to halt or reverse the loss of hearing in a person suffering from age-related hearing loss. In specific embodiments, the therapeutically effective amount of the therapeutic composition is that amount sufficient to at least partially restore hearing to the person suffering from age-related hearing loss.

In some embodiments, therapeutic compositions for the treatment or prevention of presbycusis according to the invention comprise one or more compounds selected from the group consisting of: antioxidants and/or spin-trapping agents; N-methyl-D-aspartate (NMDA) antagonists; selective serotonin reuptake inhibitor (SSRI)/NMDA antagonists; dopamine releaser/NMDA antagonists; acetylcholine release inducer/antioxidant/NMDA antagonist/norepinephrine-epinephrine reuptake inhibitors; monamineoxidase-A/serotonin reuptake inhibiting/antioxidants; norepinephrine and serotonin reuptake inhibitor/low-affinity NMDA antagonists; calcium channel antagonists and SSRI or norepinephrine selective reuptake inhibitor (NSRI); 5HT serotonin reuptake inhibiting, norepinephrine reuptake inhibiting, acetylcholine releasing and N-methyl-D-aspartate antagonists.

In some particular embodiments, therapeutic compositions for the treatment or prevention of presbycusis according to the invention comprise one or more antioxidants and/or spin-trapping agents selected from the group consisting of: allopurinol, glutathione, methionine, L-carnitine, and combinations, pharmaceutically acceptable salts and polymorphs thereof.

In other particular embodiments, therapeutic compositions for the treatment or prevention of presbycusis according to the invention comprise one or more N-methyl-D-aspartate (NMDA) antagonists selected from the group consisting of: riluzole, caroverine, memantine, magnesium and combinations, pharmaceutically acceptable salts and polymorphs thereof.

In further particular embodiments, therapeutic compositions for the treatment or prevention of presbycusis according to the invention comprise one or more selective serotonin reuptake inhibitor (SSRI)/NMDA antagonists selected from the group consisting of: alaproclate and combinations of one or more SSRIs with one or more NMDA antagonists. Suitable SSRIs include fluoxetine, sertraline, S-citalopram and combinations thereof. Suitable NMDA antagonists include riluzole, caroverine, memantine and combinations, pharmaceutically acceptable salts and polymorphs thereof.

In still further particular embodiments, therapeutic compositions for the treatment or prevention of presbycusis according to the invention comprise one or more dopamine releaser/NMDA antagonists selected from the group consisting of: amantadine and one or more combinations of compounds selected from: amantadine and zonisamide; amantadine and a SSRI; amantadine and an antioxidant; amantadine, zonisamide and a SSRI; amantadine, zonisamide and an antioxidant; amantadine, a SSRI and an antioxidant; and amantadine, zonisamide, a SSRI and an antioxidant, and pharmaceutically acceptable salts, polymorphs and combinations thereof.

In particular embodiments, therapeutic compositions for the treatment or prevention of presbycusis according to the invention comprise one or more acetylcholine release inducer/antioxidant/NMDA antagonist/norepinephrine-epinephrine reuptake inhibitors selected from the group consisting of: bifemelane, and pharmaceutically acceptable salts and/or polymorphs thereof.

In particular embodiments, therapeutic compositions for the treatment or prevention of presbycusis according to the invention comprise one or more monamineoxidase-A/serotonin reuptake inhibiting/antioxidants selected from the group consisting of: pirlindole, and pharmaceutically acceptable salts and/or polymorphs thereof.

In particular embodiments, therapeutic compositions for the treatment or prevention of presbycusis according to the invention comprise one or more norepinephrine and serotonin reuptake inhibitor/low-affinity NMDA antagonists selected from the group consisting of: milnacipran, bicifadine, and pharmaceutically acceptable salts, polymorphs and combinations thereof.

In particular embodiments, therapeutic compositions for the treatment or prevention of presbycusis according to the invention comprise one or more calcium channel antagonists and SSRI or norepinephrine selective reuptake inhibitors (NSRIs); selected from the group consisting of: nimodipine, verapamil and pharmaceutically acceptable salts, polymorphs and combinations thereof.

In particular embodiments, therapeutic compositions for the treatment or prevention of presbycusis according to the invention comprise one or more 5HT serotonin reuptake inhibiting, norepinephrine reuptake inhibiting, acetylcholine releasing and N-methyl-D-aspartate antagonists; selected from the group consisting of: indeloxazine, a combination of indeloxazine with at least one NMDA antagonist, such as riluzole, caroverine, memantine, magnesium, and pharmaceutically acceptable salts, polymorphs and combinations thereof.

D. Treatment Strategies

The invention provides methods of treating or preventing a hearing disorders. In some embodiments, the invention provides a method of treating or preventing noise-induced hearing loss, tinnitus, transmission of abnormal sounds and auditory sensations associated with tinnitus, fluid accumulation in the inner ear, facilitating central auditory processing of sounds and speech, or combinations thereof, comprising administering zonisamide to a mammal in need of such treatment in an amount sufficient to protect against noise-induced hearing loss, reduce transmission of abnormal sounds and auditory sensations associated with tinnitus, reduce fluid accumulation in the inner ear and/or facilitate central auditory processing of sounds and speech. Unless otherwise specified, treatment of one or more of the above hearing disorders is not exclusive of treatment of one or more additional hearing disorders. Moreover, treatment of a hearing disorder is not exclusive of prevention of the same or another hearing disorder, nor is prevention of a hearing disorder exclusive of treatment of the same or another hearing disorder, unless otherwise specified.

In particular embodiments, the invention provides methods of protecting against noise-induced damage or loss of hair cells in the inner cochlea of the inner ear. The method comprises administering zonisamide alone or in combination with one or more additional active ingredients to a mammal in need of protection from noise-induced hearing loss in an amount sufficient to protect against noise-induced hearing loss. In some embodiments, zonisamide is combined with at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof, in the same dosage form. In other embodiments, zonisamide is administered to the mammal in a dosage form separate from at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof.

In other embodiments, the invention provides methods of reducing transmission of abnormal sounds and auditory sensations associated with tinnitus. The method comprises administering zonisamide alone or in combination with one or more additional active ingredients to a mammal in need of reducing transmission of abnormal sounds and auditory sensations associated with tinnitus in an amount sufficient to reduce transmission of abnormal sounds and auditory sensations associated with tinnitus. In some embodiments, zonisamide is combined with at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof, in the same dosage form. In other embodiments, zonisamide is administered to the mammal in a dosage form separate from at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof.

In other embodiments, the invention provides methods of reducing fluid accumulation associated with trauma and other disorders of the inner ear. The method comprises administering zonisamide alone or in combination with one or more additional active ingredients to a mammal in need of reducing fluid accumulation associated with trauma and other disorders of the inner ear in an amount sufficient to reduce fluid accumulation. In some embodiments, zonisamide is combined with at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof, in the same dosage form. In other embodiments, zonisamide is administered to the mammal in a dosage form separate from at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof.

In other embodiments, the invention provides methods of stimulating central nervous system serotonin neurotransmission, thereby facilitating central auditory processing of sounds and speech. The method comprises administering zonisamide alone or in combination with one or more additional active ingredients to a mammal in need of such treatment in an amount sufficient to facilitate central auditory processing. In some embodiments, zonisamide is combined with at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof, in the same dosage form. In other embodiments, zonisamide is administered to the mammal in a dosage form separate from at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof.

Thus, the invention provides both methods of protecting against hearing loss and methods of treating hearing loss. In this context, protecting against hearing loss means that the active pharmaceutical ingredient or ingredients protect, at least to some degree, against the loss of hearing in a mammal. Such protection may range from slight to nearly complete. The mammal treated may be one that has already experienced hearing loss, including one that has already experienced hearing loss and is expected to be subjected to conditions similar to those that brought about the current degree of hearing loss. The mammal treated may also be one that has yet to experience notable hearing loss but is expected to be at risk for hearing loss, due to genetic profiling, expected exposure to one or more hearing-loss inducing causes (such as excessive noise), or a combination of those factors.

In the context of the methods according to the invention, treatment of hearing loss means restoring (at least in part) hearing to the mammal, or ameliorating one or more symptoms of hearing loss. Symptoms of hearing loss include experiencing abnormal sounds and auditory sensations associated with tinnitus and reduced ability to distinguish sounds and/or spoken words.

The treated mammal may be human or a non-human mammal such as a dog, a cat, a monkey, an ape, a gerbil, a hamster, a mouse, a rat, a horse, a cow, a rabbit or other mammal. It is expected that, while the dosing and other considerations may change from species to species, the person of skill in the art will be able to adapt the disclosed zonisamide treatment regimes to treat a variety of mammalian species that are currently experiencing hearing loss or are expected to face the threat of experiencing hearing loss.

In particular embodiments, the invention provides methods of protecting against noise-induced damage or loss of hair cells in the inner cochlea of the inner ear. The method comprises administering zonisamide alone or in combination with one or more additional active ingredients to a mammal in need of protection from noise-induced hearing loss in an amount sufficient to protect against noise-induced hearing loss. In some embodiments, zonisamide is combined with at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof, in the same dosage form. In other embodiments, zonisamide is administered to the mammal in a dosage form separate from at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof.

In other embodiments, the invention provides methods of reducing transmission of abnormal sounds and auditory sensations associated with tinnitus. The method comprises administering zonisamide alone or in combination with one or more additional active ingredients to a mammal in need of reducing transmission of abnormal sounds and auditory sensations associated with tinnitus in an amount sufficient to reduce transmission of abnormal sounds and auditory sensations associated with tinnitus. In some embodiments, zonisamide is combined with at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof, in the same dosage form. In other embodiments, zonisamide is administered to the mammal in a dosage form separate from at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof.

In other embodiments, the invention provides methods of reducing fluid accumulation associated with trauma and other disorders of the inner ear. The method comprises administering zonisamide alone or in combination with one or more additional active ingredients to a mammal in need of reducing fluid accumulation associated with trauma and other disorders of the inner ear in an amount sufficient to reduce fluid accumulation. In some embodiments, zonisamide is combined with at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof, in the same dosage form. In other embodiments, zonisamide is administered to the mammal in a dosage form separate from at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof.

In other embodiments, the invention provides methods of stimulating central nervous system serotonin neurotransmission, thereby facilitating central auditory processing of sounds and speech. The method comprises administering zonisamide alone or in combination with one or more additional active ingredients to a mammal in need of such treatment in an amount sufficient to facilitate central auditory processing. In some embodiments, zonisamide is combined with at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof, in the same dosage form. In other embodiments, zonisamide is administered to the mammal in a dosage form separate from at least one other active pharmaceutical ingredient, such as an anti-oxidant or spin trapping agent, an NMDA antagonist, a combination of an SSRI and a NMDA antagonist, an agent having both SSRI and NMDA antagonist activity or combinations thereof.

Thus, the invention provides both methods of protecting against hearing loss and methods of treating hearing loss. In this context, protecting against hearing loss means that the active pharmaceutical ingredient or ingredients protect, at least to some degree, against the loss of hearing in a mammal. Such protection may range from slight to nearly complete. The mammal treated may be one that has already experienced hearing loss, including one that has already experienced hearing loss and is expected to be subjected to conditions similar to those that brought about the current degree of hearing loss. The mammal treated may also be one that has yet to experience notable hearing loss but is expected to be at risk for hearing loss, due to genetic profiling, expected exposure to one or more hearing-loss inducing causes (such as excessive noise), or a combination of those factors.

In the context of the methods according to the invention, treatment of hearing loss means restoring (at least in part) hearing to the mammal, or ameliorating one or more symptoms of hearing loss. Symptoms of hearing loss include experiencing abnormal sounds and auditory sensations associated with tinnitus and reduced ability to distinguish sounds and/or spoken words.

The treated mammal may be human or a non-human mammal such as a dog, a cat, a monkey, an ape, a gerbil, a hamster, a mouse, a rat, a horse, a cow, a rabbit or other mammal. It is expected that, while the dosing and other considerations may change from species to species, the person of skill in the art will be able to adapt the disclosed zonisamide treatment regimes to treat a variety of mammalian species that are currently experiencing hearing loss or are expected to face the threat of experiencing hearing loss.

1. Zonisamide as API

The invention provides methods of preventing or treating a hearing disorder with zonisamide. The method provides administering to a mammal a therapeutic amount of zonisamide. In particular, the invention provides methods of providing protection against noise-induced loss and damage to hair cells within the cochlea of the inner ear, comprising administering a therapeutic amount of zonisamide to a mammal in need of such treatment. Zonisamide's calcium channel antagonism provides protection against noise-induced loss and damage of hair cells within the cochlea of the inner ear. Zonisamide's sodium channel blocking activity reduces transmission of the abnormal sounds and auditory sensations associated with tinnitus. Zonisamide's carbonic anhydrase inhibitory activity helps to reduce fluid accumulation associated with trauma and other disorders of the inner ear. Zonisamide's ability to stimulate central nervous system serotonin neurotransmission facilitates central auditory processing of sounds and speech.

In particular embodiments, the invention provides methods of protecting against noise-induced hearing loss, reducing transmission of abnormal sounds and auditory sensations associated with tinnitus, reducing fluid accumulation in the inner ear and/or facilitating central auditory processing of sounds and speech, comprising administering to a mammal in need of such treatment an amount of zonisamide sufficient to protect against noise-induced hearing loss, reducing transmission of abnormal sounds and auditory sensations associated with tinnitus, reducing fluid accumulation in the inner ear and/or facilitating central auditory processing of sounds and speech.

2. Zonisamide and Antioxidants or Spin Trapping Agents

In some embodiments, the invention provides a method of protecting against noise-induced hearing loss, reducing transmission of abnormal sounds and auditory sensations associated with tinnitus, reducing fluid accumulation in the inner ear, facilitating central auditory processing of sounds and speech, or combinations thereof, comprising administering zonisamide and one or more active pharmaceutical agents that bind to or metabolize reactive oxygen species and provide protection against the damage induced by oxygen species, which are toxic mediators. In some such embodiments, the zonisamide is administered in combination with an antioxidant or spin trapping agent. In some such embodiments, zonisamide is administered in combination with allopurinol, methionine or L-camitine. In particular embodiments, zonisamide is administered in combination with allopurinol. In other particular embodiments, zonisamide is administered in combination with glutathione. In still further particular embodiments, zonisamide is administered in combination with methionine. In yet further embodiments, zonisamide is administered in combination with L-carnitine. In yet further embodiments, zonisamide is administered in combination with two or more antioxidants, such as allopurinol, glutathione, methionine, or L-carnitine. In still further embodiments, zonisamide is administered in combination with one or more antioxidants, such as allopurinol, glutathione, methionine, or L-carnitine, and one or more other active pharmaceutical ingredients, such as one or more NMDA antagonists, one or more SSRIs or one or more compounds having both SSRI and NMDA antagonist activity, such as alaproclate (2-(p-chlorophenyl)-1,1-dimethyl 2-aminopropanoate).

In some embodiments, zonisamide is administered in the same dosage form as one or more antioxidants or spin trapping agents. In some such embodiments, the zonisamide is mixed with one or more antioxidants or spin trapping agents. In others, the zonisamide is segregate from the antioxidant or spin trapping agent by a coating, a shell, a capsule or some other means for preventing admixture of zonisamide with the antioxidant or spin trapping agent, while maintaining both ingredients in the same dosage form.

3. Zonisamide and NMDA Antagonists

In some embodiments, the invention provides a method of protecting against noise-induced hearing loss, reducing transmission of abnormal sounds and auditory sensations associated with tinnitus, reducing fluid accumulation in the inner ear, facilitating central auditory processing of sounds and speech, or combinations thereof, comprising administering zonisamide and one or more active pharmaceutical agents that block the excitotoxic actions of glutamate within the inner ear. Glutamate is a mediator of noise-induced damage to the hair cells of the inner ear and blocking N-methyl-D-aspartate (NMDA) receptors provides protection against the toxic effects of glutamate. In some embodiments, zonisamide is administered in a single dosage form comprising zonisamide and a NMDA antagonist.

In some such embodiments, zonisamide is administered in a single dosage form comprising an antagonist of N-methyl-D-aspartate, such as magnesium, riluzole, caroverine, memantine or a combination of two or more thereof. In particular embodiments, zonisamide is administered in a single dosage form comprising riluzole. In other particular embodiments, zonisamide is administered in a single dosage form comprising caroverine. In still further particular embodiments, zonisamide is administered in a single dosage form comprising memantine. In still further particular embodiments, zonisamide is administered in a single dosage form comprising magnesium. In yet further embodiments, zonisamide is administered in a dosage form comprising two or more NMDA antagonists, such as magnesium, riluzole, caroverine, or memantine. In still further embodiments, zonisamide is administered in a single dosage form comprising one or more NMDA antagonists, such as magnesium, riluzole, caroverine, or memantine, and one or more other active pharmaceutical ingredients, such as one or more antioxidants or spin trapping agents, one or more SSRIs or one or more compounds having both SSRI and NMDA antagonist activity.

In some such embodiments, zonisamide is administered in a dosage form separate from that containing an NMDA antagonist, such as magnesium, riluzole, caroverine, memantine or a combination of two or more thereof. In particular embodiments, zonisamide is administered in one dose and magnesium is administered in a separate dose. In particular embodiments, zonisamide is administered in one dose and riluzole is administered in a separate dose. In other particular embodiments, zonisamide is administered in one dose and caroverine is administered in a separate dose. In still further particular embodiments, zonisamide is administered in one dose and memantine is administered in another dose. In yet further embodiments, zonisamide is administered in one dose and two or more NMDA antagonists, such as magnesium, riluzole, caroverine, or memantine are administered in a separate dose. In still further embodiments, zonisamide is administered one dose and one or more NMDA antagonists, such as magnesium, riluzole, caroverine, or memantine, and one or more other active pharmaceutical ingredients, such as one or more antioxidants or spin trapping agents, one or more SSRIs or one or more compounds having both SSRI and NMDA antagonist activity, are administered in a separate dose.

As mentioned above, in some embodiments, zonisamide is administered in the same dosage form as one or more NMDA antagonists. In some such embodiments, the zonisamide is mixed directly with one or more NMDA antagonists. In other embodiments, the zonisamide is segregate from one or more NMDA antagonists by a coating, a shell, a capsule or some other means for preventing admixture of zonisamide with the antioxidant or spin trapping agent, while maintaining both ingredients in the same dosage form.

4. Zonisamide and SSRI/NMDA Antagonists

In some embodiments, the invention provides a method of protecting against noise-induced hearing loss, reducing transmission of abnormal sounds and auditory sensations associated with tinnitus, reducing fluid accumulation in the inner ear, facilitating central auditory processing of sounds and speech, or combinations thereof, comprising administering zonisamide and one or more active pharmaceutical agents that enhance synaptic levels of serotonin in the brain and enhance hearing by improving auditory processing, increasing the signal: noise ratio of environmental sounds, and/or by heightening attention.

In some such embodiments, zonisamide is administered in combination with a selective serotonin reuptake inhibitor (SSRI). In some such embodiments, zonisamide is administered in combination with fluoxetine, sertraline, S-citalopram or combinations thereof. In particular embodiments, zonisamide is administered in combination with fluoxetine. In other particular embodiments, zonisamide is administered in combination with sertraline. In still further particular embodiments, zonisamide is administered in combination with S-citalopram. In still further embodiments, zonisamide is administered in combination with two or more SSRI agents, such as fluoxetine, sertraline or S-citalopram.

In some advantageous embodiments, zonisamide is administered in combination with at least one SSRI and at least one NMDA antagonist. In exemplary embodiments, zonisamide is administered in combination with at least one SSRI (such as fluoxetine, sertraline, S-citalopram or combinations of two or more thereof) and one or more NMDA antagonists selected from magnesium, riluzole, caroverine and memantine. In some particular embodiments, zonisamide is administered in combination with at least one SSRI (such as fluoxetine, sertraline, S-citalopram or combinations of two or more thereof) and magnesium. In some particular embodiments, zonisamide is administered in combination with at least one SSRI (such as fluoxetine, sertraline, S-citalopram or combinations of two or more thereof) and riluzole. In other particular embodiments, zonisamide is administered in combination with at least one SSRI (such as fluoxetine, sertraline, S-citalopram or combinations of two or more thereof) and caroverine. In yet other embodiments, zonisamide is administered in combination with at least one SSRI (such as fluoxetine, sertraline, S-citalopram or combinations of two or more thereof) and memantine. In yet further embodiments, zonisamide is administered in combination with at least one SSRI (such as fluoxetine, sertraline, S-citalopram or combinations of two or more thereof) and a combination of two or more of magnesium, riluzole, caroverine and memantine.

In some embodiments, zonisamide is administered in a combination comprising at least one at least one agent having combined SSRI and NMDA antagonist activity.

In some embodiments, zonisamide is administered in a dosage form comprising at least one agent having both SSRI and NMDA antagonist activity or at least one SSRI and at least one NMDA antagonist. In some such embodiments, the dosage form further comprises at least one antioxidant or spin trapping agent.

In some embodiments, zonisamide is administered in a dosage form separate from at least one agent having both SSRI and NMDA antagonist activity or zonisamide is administered in a dosage form separate from at least one SSRI or at least one NMDA antagonist. In some such embodiments, the zonisamide is mixed with one or more at least one agent having both SSRI and NMDA antagonist activity or zonisamide is mixed with at least one SSRI or at least one NMDA antagonist. In other embodiments, the zonisamide is segregate from the SSRI/NMDA antagonist, SSRI or NMDA antagonist by a coating, a shell, a capsule or some other means for preventing admixture of zonisamide and the other active pharmaceutical ingredient, while maintaining the ingredients in the same dosage form.

5. Amantadine

The invention provides methods of preventing or treating a hearing disorder in a mammal, such as a human, using amantadine. Amantadine is a dopamine releaser and a N-methyl-D-aspartate antagonist. The dopamine releasing effect of amantadine will enhance auditory processing, while the NMDA antagonistic effect will protect inner ear hair cells from glutamate-induced toxicity. Thus, the invention provides a method of preventing or treating hearing loss in a mammal, such as a human, comprising administering a therapeutic amount of amantadine to the mammal. Additionally, the invention provides a method of preventing or treating an auditory disorder, such as tinnitus, comprising administering a therapeutic amount of amantadine to the mammal. Also, the invention provides a method of preventing or treating hearing loss and tinnitus, comprising administering to a mammal a therapeutic amount of amantadine.

6. Amantadine and Zonisamide

The invention provides methods of preventing or treating a hearing disorder in a mammal, such as a human, using amantadine. Amantadine is a dopamine releaser and a N-methyl-D-aspartate antagonist. The dopamine releasing effect of amantadine will enhance auditory processing, while the NMDA antagonistic effect will protect inner ear hair cells from glutamate-induced toxicity. Zonisamide's calcium channel antagonist activity will provide protection against noise-induced loss and damage of hair cells within the cochlea of the inner ear. Zonisamide's sodium channel blocking activity will reduce transmission of the abnormal sounds and auditory sensations associated with tinnitus. Zonisamide's carbonic anhydrase inhibitory activity helps to reduce fluid accumulation associated with trauma and other disorders of the inner ear. Finally, zonisamide's ability to stimulate central nervous system serotonin neurotransmission facilitates central auditory processing of sounds and speech. Thus, the invention provides a method of preventing or treating hearing loss, an auditory disorder such as tinnitus, or both in a mammal, such as a human, comprising administering a therapeutic amount of a combination of amantadine and zonisamide to the mammal.

In some embodiments, the method comprises administering to the mammal amantadine and zonisamide are combined in the same dosage form. In particular embodiments, amantadine and zonisamide mixed together. In other embodiments, amantadine and zonisamide are combined with one or more excipients to form a biphasic dosage form, wherein amantadine and zonisamide occupy separate phases.

In other embodiments, amantadine and zonisamide are administered in separate dosage forms. In particular embodiments, the separate dosage forms are administered simultaneously or substantially simultaneously (e.g. within about 10 minutes of one another, more particularly within about 5 minutes of one another, even more particularly within about 2 minutes of one another). In other embodiments, the separate dosage forms are administered at substantially different times (e.g. more than about 10 minutes apart, more particularly more than about an hour apart). The dosage forms include those that are currently or presently commercially available, as well as those available to the person having skill in the art. They include tablets, capsules, caplets, gel caps, powders, solutions, sols, etc.

In some embodiments, the separate dosage forms of amantadine and zonisamide are provided in a kit, such as is defined in more detail below. In specific embodiments, the separate dosages are provided in a kit including instructions for the administration of amantadine and zonisamide for the prevention or treatment of hearing disorders, especially for the prevention or treatment of hearing loss, tinnitus or both.

7. Amantadine and SSRI

The invention provides methods of preventing or treating a hearing disorder in a mammal, such as a human, using amantadine in combination with a selective serotonin reuptake inhibitor. Amantadine is a dopamine releaser and a N-methyl-D-aspartate antagonist. The dopamine releasing effect of amantadine will enhance auditory processing, while the NMDA antagonistic effect will protect inner ear hair cells from glutamate-induced toxicity. In some such embodiments, amantadine is administered in combination with a selective serotonin reuptake inhibitor, such as fluoxetine, sertraline, S-citalopram or combinations thereof. In particular embodiments, amantadine is administered in combination with fluoxetine. In other particular embodiments, amantadine is administered in combination with sertraline. In still further particular embodiments, amantadine is administered in combination with S-citalopram. hi still further embodiments, amantadine is administered in combination with two or more SSRI agents, such as fluoxetine, sertraline or S-citalopram.

Thus, the invention provides a method of preventing or treating hearing loss in a mammal, such as a human, comprising administering a therapeutic amount of a combination of amantadine and one or more selective serotonin reuptake inhibitors to the mammal.

In some embodiments, the method comprises administering to the mammal amantadine and selective serotonin reuptake inhibitor are combined in the same dosage form. In particular embodiments, amantadine and selective serotonin reuptake inhibitor mixed together. In other embodiments, amantadine and selective serotonin reuptake inhibitor are combined with one or more excipients to form a biphasic dosage form, wherein amantadine and selective serotonin reuptake inhibitor occupy separate phases.

In other embodiments, amantadine and selective serotonin reuptake inhibitor are administered in separate dosage forms. In particular embodiments, the separate dosage forms are administered simultaneously or substantially simultaneously (e.g. within about 10 minutes of one another, more particularly within about 5 minutes of one another, even more particularly within about 2 minutes of one another). In other embodiments, the separate dosage forms are administered at substantially different times (e.g. more than about 10 minutes apart, more particularly more than about an hour apart). The dosage forms include those that are currently or presently commercially available, as well as those available to the person having skill in the art. They include tablets, capsules, caplets, gel caps, powders, solutions, sols, etc.

In some embodiments, the separate dosage forms of amantadine and selective serotonin reuptake inhibitor are provided in a kit, such as is defined in more detail below. In specific embodiments, the separate dosages are provided in a kit including instructions for the administration of amantadine and selective serotonin reuptake inhibitor for the prevention or treatment of hearing disorders, especially for the prevention or treatment of hearing loss, tinnitus or both.

8. Amantadine and Antioxidant

The invention provides methods of preventing or treating a hearing disorder in a mammal, such as a human, using amantadine in combination with an antioxidant. Amantadine is a dopamine releaser and a N-methyl-D-aspartate antagonist. The dopamine releasing effect of amantadine will enhance auditory processing, while the NMDA antagonistic effect will protect inner ear hair cells from glutamate-induced toxicity. In some such embodiments, amantadine is administered in combination with an antioxidant, such as allopurinol, methionine or L-carnitine. In particular embodiments, amantadine is administered in combination with allopurinol. In other particular embodiments, amantadine is administered in combination with glutathione. In still further particular embodiments, amantadine is administered in combination with methionine. In yet further embodiments, amantadine is administered in combination with L-carnitine. In yet further embodiments, amantadine is administered in combination with two or more antioxidants, such as allopurinol, glutathione, methionine, or L-carnitine.

Thus, the invention provides a method of preventing or treating hearing loss in a mammal, such as a human. The method comprises administering a therapeutic amount of a combination of amantadine and one or more antioxidants, such as allopurinol, glutathione, methionine or L-carnitine.

In some embodiments, the method comprises administering to the mammal amantadine and antioxidant are combined in the same dosage form. In particular embodiments, amantadine and antioxidant are mixed together. In other embodiments, amantadine and antioxidant are combined with one or more excipients to form a biphasic dosage form, wherein amantadine and antioxidant occupy separate phases.

In other embodiments, amantadine and antioxidant are administered in separate dosage forms. In particular embodiments, the separate dosage forms are administered simultaneously or substantially simultaneously (e.g. within about 10 minutes of one another, more particularly within about 5 minutes of one another, even more particularly within about 2 minutes of one another). In other embodiments, the separate dosage forms are administered at substantially different times (e.g. more than about 10 minutes apart, more particularly more than about an hour apart). The dosage forms include those that are currently or presently commercially available, as well as those available to the person having skill in the art. They include tablets, capsules, caplets, gel caps, powders, solutions, sols, etc.

In some embodiments, the separate dosage forms of amantadine and antioxidant are provided in a kit, such as is defined in more detail below. In specific embodiments, the separate dosages are provided in a kit including instructions for the administration of amantadine and antioxidant for the prevention or treatment of hearing disorders, especially for the prevention or treatment of hearing loss, tinnitus or both.

9. Bifemelane

The invention provides methods of preventing or treating a hearing disorder in a mammal, such as a human, using bifemelane. Bifemelane is an acetylcholine release inducer, an antioxidant, a N-methyl-D-aspartate antagonist and a norepinephrine reuptake inhibitor. The ability of bifemelane to enhance brain levels of acetycholine and norepinephrine improves auditory processing, speech recognition and hearing perception. The ability of bifemelane to block N-methyl-D-aspartate receptors and to act as an antioxidant provides protection to the inner ear cells. Thus, the invention provides a method of preventing or treating hearing loss, an auditory disorder, such as tinnitus, or both in a mammal, such as a human. The method comprises administering a therapeutic amount of bifemelane to the mammal.

10. Pirlindole

The invention provides methods of preventing or treating a hearing disorder in a mammal, such as a human, using pirlindole, which is a monamineoxidase-A inhibitor, a serotonin reuptake inhibitor and an antioxidant. Pirlindole's central effects via increasing norepinephrine and serotonin enhance auditory processing. The antioxidant activity of pirlindole provide protection to inner ear hair cells from damage caused by reactive oxidative species. Thus, the invention provides a method of preventing or treating hearing loss, an auditory disorder, such as tinnitus, or both in a mammal, such as a human. The method comprises administering a therapeutic amount of pirlindole to the mammal.

11. Pirlindole and NMDA Antagonist

The invention provides methods of preventing or treating a hearing disorder in a mammal, such as a human, using pirlindole and an antagonist of N-methyl-D-aspartate. Pirlindole is a monamineoxidase-A inhibitor, a serotonin reuptake inhibitor and an antioxidant. Pirlindole's central effects via increasing norepinephrine and serotonin enhance auditory processing. The antioxidant activity of pirlindole provides protection to inner ear hair cells from damage caused by reactive oxidative species. Antagonists of N-methyl-D-aspartate, such as magnesium, riluzole, caroverine and memantine, provide protection against the toxic effects of glutamate, thereby protecting the hair cells of the cochlea of the inner ear from noise-induced damage.

Thus, the invention provides a method of preventing or treating hearing loss, an auditory disorder, such as tinnitus, or both in a mammal, such as a human. The method comprises administering a therapeutic amount of a combination of pirlindole and an antagonist of N-methyl-D-aspartate to the mammal. In some embodiments, the invention provides methods of preventing or treating hearing loss, comprising administering a therapeutic amount of pirlindole and an antagonist of NMDA selected from the group consisting of magnesium, riluzole, caroverine, memantine and combinations thereof, to a mammal. In some embodiments, the methods comprise administration of a therapeutic amount of a combination of pirlindole and magnesium. In other particular embodiments, the methods comprise administration of a therapeutic amount of a combination of pirlindole and riluzole. In still further embodiments, the methods comprise administration of a therapeutic amount of a combination of pirlindole and caroverine. In other particular embodiments, the methods comprise administration of a therapeutic amount of a combination of pirlindole and memantine. In other embodiments, the methods comprise administration of a therapeutic amount of a combination of pirlindole and two or more members of the group consisting of magnesium, riluzole, caroverine and memantine.

12. Pirlindole and Amantadine

The invention provides methods of preventing or treating a hearing disorder in a mammal, such as a human, using pirlindole. Pirlindole is a monamineoxidase-A inhibitor, a serotonin reuptake inhibitor and an antioxidant. Pirlindole's central effects via increasing norepinephrine and serotonin enhance auditory processing. The antioxidant activity of pirlindole provide protection to inner ear hair cells from damage caused by reactive oxidative species. Amantadine is a dopamine releaser and a N-methyl-D-aspartate antagonist. The dopamine releasing effect of amantadine will enhance auditory processing, while the NMDA antagonistic effect will protect inner ear hair cells from glutamate-induced toxicity. Thus, the invention provides a method of preventing or treating hearing loss, an auditory disorder, such as tinnitus, or both in a mammal, such as a human. The method comprises administering a therapeutic amount of pirlindole to the mammal.

13. Zonisamide and Milnacipran or Bicifadine

The invention provides methods of preventing or treating a hearing disorder in a mammal, such as a human, using: zonisamide and milnacipran; zonisamide and bicifadine; or zonisamide, milnacipran and bicifadine. Zonisamide's sodium channel blocking activity will reduce transmission of the abnormal sounds and auditory sensations associated with tinnitus. Zonisamide's carbonic anhydrase inhibitory activity helps to reduce fluid accumulation associated with trauma and other disorders of the inner ear. Finally, zonisamide's ability to stimulate central nervous system serotonin neurotransmission facilitates central auditory processing of sounds and speech. Milnacipran and bicifadine are norepinephrine-serotonin reuptake inhibitors, weak N-methyl-D-aspartate antagonists, which improve auditory processing (NSRI activity) and provide protection to inner ear hair cells (NMDA antagonist activity). Milnacipran, bicifadine or a combination of both will provide both central and peripheral benefits to the treated mammal. Thus, the invention provides a method of preventing or treating hearing loss, an auditory disorder such as tinnitus, or both in a mammal, such as a human, comprising administering a therapeutic amount of a combination of zonisamide and a member of the group consisting of milnacipran, bicifadine or a combination of both to the mammal.

In some embodiments, the method comprises administering to the mammal zonisamide and a member of the group consisting of milnacipran, bicifadine and a combination of both in the same dosage form. In particular embodiments, zonisamide and a member of the group consisting of milnacipran, bicifadine and a combination of both are mixed together. In other embodiments, zonisamide and a member of the group consisting of milnacipran, bicifadine and a combination of both are combined with one or more excipients to form a biphasic or multiphasic dosage form, wherein zonisamide and a member of the group consisting of milnacipran, bicifadine and a combination of both occupy separate phases.

In other embodiments, zonisamide and a member of the group consisting of milnacipran, bicifadine and a combination of both are administered in separate dosage forms. In particular embodiments, the separate dosage forms are administered simultaneously or substantially simultaneously (e.g. within about 10 minutes of one another, more particularly within about 5 minutes of one another, even more particularly within about 2 minutes of one another). In other embodiments, the separate dosage forms are administered at substantially different times (e.g. more than about 10 minutes apart, more particularly more than about an hour apart). The dosage forms include those that are currently or presently commercially available, as well as those available to the person having skill in the art. They include tablets, capsules, caplets, gel caps, powders, solutions, sols, etc.

In some embodiments, the separate dosage forms of zonisamide and a member of the group consisting of milnacipran, bicifadine and a combination of both are provided in a kit, such as is defined in more detail below. In specific embodiments, the separate dosages are provided in a kit including instructions for the administration of amantadine and zonisamide for the prevention or treatment of hearing disorders, especially for the prevention or treatment of hearing loss, tinnitus or both.

14. Calcium Channel Antagonist and SSRI, NSRI or MAO-A

The invention provides methods of preventing or treating a hearing disorder with a calcium channel antagonist. The method provides administering to a mammal a therapeutic amount of a calcium channel antagonist. In particular, the invention provides methods of providing protection against noise-induced loss and damage to hair cells within the cochlea of the inner ear, comprising administering a therapeutic amount of zonisamide to a mammal in need of such treatment. Calcium channel antagonists provide protection against noise-induced loss and damage of hair cells within the cochlea of the inner ear. Selective serotonin reuptake inhibitors, norepinephrine-serotonin reuptake inhibitors and monamineoxidase-A inhibitors enhance central auditory processing.

In particular embodiments, the invention provides methods of treating or preventing a hearing disorder using a combination of a calcium channel antagonist and a selective serotonin reuptake inhibitor. Suitable calcium channel antagonists for use in the invention include nimodipine, verapamil and combinations thereof. Suitable selective serotonin reuptake inhibitors include

In particular embodiments, the invention provides methods of protecting against noise-induced hearing loss, reducing transmission of abnormal sounds and auditory sensations associated with tinnitus, reducing fluid accumulation in the inner ear and/or facilitating central auditory processing of sounds and speech, comprising administering to a mammal in need of such treatment an amount of zonisamide sufficient to protect against noise-induced hearing loss, reducing transmission of abnormal sounds and auditory sensations associated with tinnitus, reducing fluid accumulation in the inner ear and/or facilitating central auditory processing of sounds and speech.

15. Indeloxazine

The invention provides methods of preventing or treating a hearing disorder in a mammal, such as a human, using indeloxazine, which is a 5HT serotonin reuptake inhibitor, a norepinephrine reuptake inhibitor, an acetylcholine releaser, and an antagonist of N-methyl-D-aspartate. The ability of indeloxazine to increase brain serotonin, norepinephrine, and acetycholine levels improves auditory processing, speech recognition and hearing perception. The ability of indeloxazine to block N-methyl-D-aspartate receptors and to act as an antioxidant provides protection to the inner ear cells. Thus, the invention provides a method of preventing or treating hearing loss, an auditory disorder, such as tinnitus, or both in a mammal, such as a human. The method comprises administering a therapeutic amount of indeloxazine to the mammal.

17. Indeloxazine and NMDA Antagonist

The invention provides methods of preventing or treating a hearing disorder in a mammal, such as a human, using indeloxazine in combination with an antagonist of N-methyl-D-aspartate. Indeloxazine is a 5HT serotonin reuptake inhibitor, a norepinephrine reuptake inhibitor, an acetylcholine releaser, and an antagonist of N-methyl-D-aspartate. The ability of indeloxazine to increase brain serotonin, norepinephrine, and acetylcholine levels improves auditory processing, speech recognition and hearing perception. The ability of indeloxazine to block N-methyl-D-aspartate receptors and to act as an antioxidant provides protection to the inner ear cells. Blocking of the N-methyl-D-aspartate receptor provides protection to the inner ear hair cells by interfering with glutamate-mediated noise-induced damage to the hair cells of the inner ear.

In some such embodiments, indeloxazine is administered in a single dosage form comprising an antagonist of N-methyl-D-aspartate, such as magnesium, riluzole, caroverine, memantine or a combination of two or more thereof. Thus, the invention provides a method of preventing or treating hearing loss, an auditory disorder such as tinnitus, or both in a mammal, such as a human, comprising administering a therapeutic amount of a combination of indeloxazine and an antagonist of N-methyl-D-aspartate to the mammal, such as riluzole, caroverine, memantine or a combination of two or more thereof.

In some embodiments, the method comprises administering to the mammal indeloxazine and at least one antagonist of N-methyl-D-aspartate, such as magnesium, riluzole, caroverine or memantine, are combined in the same dosage form. In particular embodiments, indeloxazine and at least one antagonist of N-methyl-D-aspartate are mixed together. In other embodiments, indeloxazine and at least one antagonist of N-methyl-D-aspartate are combined with one or more excipients to form a biphasic dosage form, indeloxazine and at least one antagonist of N-methyl-D-aspartate are occupy separate phases.

In other embodiments, indeloxazine and at least one antagonist of N-methyl-D-aspartate, such as magnesium, riluzole, caroverine or memantine, are administered in separate dosage forms. In particular embodiments, the separate dosage forms are administered simultaneously or substantially simultaneously (e.g. within about 10 minutes of one another, more particularly within about 5 minutes of one another, even more particularly within about 2 minutes of one another). In other embodiments, the separate dosage forms are administered at substantially different times (e.g. more than about 10 minutes apart, more particularly more than about an hour apart). The dosage forms include those that are currently or presently commercially available, as well as those available to the person having skill in the art. They include tablets, capsules, caplets, gel caps, powders, solutions, sols, etc.

In some embodiments, the separate dosage forms of indeloxazine and at least one antagonist of N-methyl-D-aspartate, such as magnesium, riluzole, caroverine or memantine, are provided in a kit, such as is described in more detail below. In specific embodiments, the separate dosages are provided in a kit including instructions for the administration of indeloxazine and at least one antagonist of N-methyl-D-aspartate are administered for the prevention or treatment of hearing disorders, especially for the prevention or treatment of hearing loss, tinnitus or both.

It is understood that the disclosed methods are not limited to the particular methodology, protocols, and reagents described as these may vary. It is also to be understood that the terminology used herein is for the purpose of describing particular embodiments only, and is not intended to limit the scope of the present invention which will be limited only by the appended claims.

IV. Kits

As discussed above, in some embodiments, two or more active pharmaceutical ingredients may be co-administered to a mammal for the prevention or treatment of hearing disorders. In particular, zonisamide may be co-administered with or more additional active pharmaceutical ingredients, such as amantadine, milnacipran, bicifadine, antioxidants or spin trapping agents, NMDA antagonists, SSRI NMDA antagonist compounds, or combinations of at least one SSRI and at least one NMDA antagonist. In other embodiments, pirlindole may be co-administered along with an antagonist of NMDA or amantadine for the prevention or treatment of a hearing disorder. In still further embodiments, a calcium channel antagonist may be co-administered along with a SSRI or a NSRI. In further embodiments, indeloxazine may be used in combination with an antagonist of NMDA. In such cases, it is advantageous to make the specific drug combination available in the form of a kit.

In some embodiments, the invention provides a kit including zonisamide in a dosage form, especially a dosage form for oral administration. In particular, zonisamide is marketed by DAINIPPON under the trade name Zonegran™ in 25, 50 and 100 mg capsules for oral administration. Thus, in some embodiments of the invention, the kit includes one or more doses of zonisamide in capsules. In other embodiments, however, the doses of zonisamide may be present in a variety of dosage forms, such as tablets, caplets, gel caps, powders for suspension, etc.

A kit according to the invention includes at least two dosage forms, one comprising a first active pharmaceutical ingredient and the other comprising at least a second active pharmaceutical ingredient, other than the first active pharmaceutical ingredient. In some embodiments, the kit includes sufficient doses for a period of time. In particular embodiments, the kit includes a sufficient dose of each active pharmaceutical ingredient for a day, a week, 14 days, 28 days, 30 days, 90 days, 180 days, a year, etc. In some specific embodiments, the each dose is physically separated into a compartment, in which each dose is segregated from the others.

In some embodiments, the kit according to the invention includes at least two dosage forms, one comprising zonisamide and the other comprising at least one active pharmaceutical ingredient other than zonisamide. In some embodiments, the kit includes sufficient doses for a period of time. In particular embodiments, the kit includes a sufficient dose of each active pharmaceutical ingredient for a day, a week, 14 days, 28 days, 30 days, 90 days, 180 days, a year, etc. In some specific embodiments, the each dose is physically separated into a compartment, in which each dose is segregated from the others.

In particular embodiments, the kit may advantageously be a blister pack. Blister packs are known in the art, and generally include a clear side having compartments (blisters or bubbles), which separately hold the various doses, and a backing, such as a paper, foil, paper-foil or other backing, which is easily removed so that each dose may be separately extracted from the blister pack without disturbing the other doses. In some embodiments, the kit may be a blister pack in which each day's dose of a first active pharmaceutical ingredient and at least a second active pharmaceutical ingredient are segregated from the other doses in separate blisters or bubbles. In some such embodiments, the blister pack may have perforations, which allow each daily dose to be separated from the others by tearing it away from the rest of the blister pack. The separate dosage forms may be contained within separate blisters. Segregation of the two active pharmaceutical ingredients into separate blisters can be advantageous in that it prevents separate dosage forms (e.g. tablet and capsule) from contacting and damaging one another during shipping and handling. Additionally, the separate dosage forms can be accessed and/or labeled for administration to the patient at different times.

In some embodiments, the kit may be a blister pack in which each day's dose of zonisamide and at least one other active pharmaceutical ingredient is segregated from the other doses in separate blisters or bubbles. In some such embodiments, the blister pack may have perforations, which allow each daily dose to be separated from the others by tearing it away from the rest of the blister pack. The separate dosage forms may be contained within separate blisters. For example, when zonisamide is to be co-administered with riluzole, a specific number of daily doses may be divided into separate removable daily segments, each segment having at least blister containing zonisamide (e.g. a 25, 50 or 100 mg capsule of zonisamide) and at least one other blister containing riluzole (e.g. a 50 mg tablet of riluzole), with perforations separating the segment from its neighbor or neighbors. Segregation of the two active pharmaceutical ingredients into separate blisters can be advantageous in that it prevents separate dosage forms (e.g. tablet and capsule) from contacting and damaging one another during shipping and handling. Additionally, the separate dosage forms can be accessed and/or labeled for administration to the patient at different times. For example, zonisamide may cause drowsiness in some patients, and so may be labeled for nighttime administration, whereas other active pharmaceutical ingredients may promote alertness and so may be labeled for daytime administration.

In other embodiments, the kit may be box having separate compartments with separate lids. For example, a kit may comprise a box having seven compartments, each for a separate day of the week, and each compartment marked to indicate which day of the week it corresponds to. In some specific embodiments, each compartment is further subdivided to permit segregation of one active pharmaceutical ingredient from another. As stated above, such segregation is advantageous in that it prevents damage to the dosage forms and permits dosing at different times and labeling to that effect.

It will be understood that kits according to the present invention include those in which the first active pharmaceutical ingredient is selected from the group consisting of zonisamide, amantadine, pirlindole, indeloxazine, calcium channel antagonists, and the second active pharmaceutical ingredient is selected from the group consisting of antioxidants, SSRIs, NSRIs, and antagonists of NMDA. Particular kits combine zonisamide and one or members of the group consisting of allopurinol, glutathione, methionine and L-carnitine, riluzole, caroverine, memantine, magnesium, fluoxetine, sertraline, S-citalopram, alaproclate, milnacipran, bicifadine, nimodipine or verapamil. Other kits combine amantadine and one or members of the group consisting of allopurinol, glutathione, methionine and L-carnitine, riluzole, caroverine, memantine, magnesium, fluoxetine, sertraline, S-citalopram, alaproclate, milnacipran, bicifadine, nimodipine or verapamil. Other particular kits combine pirlindole and one or members of the group consisting of allopurinol, glutathione, methionine and L-carnitine, riluzole, caroverine, memantine, magnesium, fluoxetine, sertraline, S-citalopram, alaproclate, milnacipran, bicifadine, nimodipine or verapamil. Still further kits combine indeloxazine and one or members of the group consisting of allopurinol, glutathione, methionine and L-carnitine, riluzole, caroverine, memantine, magnesium, fluoxetine, sertraline, S-citalopram, alaproclate, milnacipran, bicifadine, nimodipine or verapamil. Other kits combine at least one calcium channel antagonist and one or members of the group consisting of allopurinol, glutathione, methionine and L-carnitine, riluzole, caroverine, memantine, magnesium, fluoxetine, sertraline, S-citalopram, alaproclate, milnacipran, bicifadine, nimodipine or verapamil.

V. Experimental Procedures for Hearing Loss Studies

The following experimental procedures are designed to demonstrate the effectiveness of zonisamide in the treatment of DIHL and NIHL.

A. Cochlear Cultures

Explants of sensory epithelium from the basal turn of postnatal day 5 (p5) Sprague-Dawley rats are isolated using the methods of Van De Water et al. (“Growth of the inner ear organ culture,” Ann. Otol. Rhinol. Lryngol. 83, 1-16 (1974)) and Sobkowicz et al. (“Tissue culture of the organ of Corti.,” Acta Otolaryngol. Suppl. 502, 3-36 (1993)). Explants are maintained in Dulbecco's Modified Eagle's medium with 10% fetal bovine serum and 30 U/ml penicillin. HEPES buffer is added to a concentration of 25 mM and the glucose concentration is increased to 6 mg/ml to enhance neuronal survival. Each 15 mm dish containing 25 μl of medium is maintained in an incubator at 37° C. with 5% CO₂ and 95% humidity.

Toxicity inducers (gentamycin or cisplatin, 1-100 μM) are exposed to the medium 12 to 24 hours following plating for 48 hours. Concentration of gentamycin or cisplatin are employed that produce approximately 50% loss of outer hair cells, which permit assessments of either increases or decreases in outer hair cell damage.

Test compounds are applied to the cultures for 12 hours, and then challenged with medium containing gentamycin or cisplatin plus the test compound for an additional 48 hours, followed by fixation and evaluation.

At the end of culture, free-floating explants are fixed with 4% paraformaldehyde and 1% glutaraldehyde in phosphate-buffered saline. The explants are then rendered permeable with 1% Tween-20 in phosphate-buffered saline. The explants are then strained with conjugated phalloidin-rhodamine probe in phosphate-buffered saline, washed twice, and mounted between two cover slips in a 1:3 mixture of phosphate-buffered saline/glycerol. Explants are imaged by light and fluorescence microscopy and by laser confocal microscopy to assess the degree of damage to the outer hair cells.

B. Animal Models

Experimental animals

Male CBA mice, at an initial age of 4 weeks, are purchased from Harlan Sprague-Dawley Co. (Indianapolis, Ind.). The animals are given free access to water and a regular mouse diet (Purina, St. Louis, Mo.), and are allowed 1 week to acclimate before treatment.

Drug-Induced Hearing Loss

The aminoglycoside antibiotic kanamycin (700 mg of kanamycin base/kg of body weight twice daily) is tested alone and in combination with various concentrations of zonisamide. The study to evaluate auditory effects is comprised of one group serving as the vehicle control group, one group receiving kanamycin injections only, three groups receiving kanamycin plus zonisamide at 0.1, 0.7, or 1.3 mg/kg twice daily, and three groups receiving zonisamide only at 0.1, 0.7, or 1.3 mg/kg twice daily. In a separate study, levels of kanamycin in serum are determined for animals receiving kanamycin alone (700 mg of kanamycin base/kg twice daily) or kanamycin plus zonisamide (0.1 or 0.7 mg/kg twice daily). Kanamycin and zonisamide are each dissolved in suitable vehicle, and injections are given separately but simultaneously twice daily for 15 days. Control mice received an equivalent volume of vehicle.

Noise-Induced Hearing Loss

Mice are exposed to a 4 kHz octave band of noise at 110 dB SPL for 4 h and receive treatment with vehicle or zonisamide (0.1, 0.7, or 1.3 mg/kg) twice daily beginning 3 days prior and continuing until 1 day following noise exposure.

Evaluation of Auditory Function

Auditory thresholds are measured by evoked auditory brainstem responses (ABR). Thresholds are taken for each animal at thebeginning of the study for all animals. For animals treated with kanamycin, ABR measurements are also assessed 2 weeks after the start of drug treatment, and then at 3 weeks and 5 weeks. For animals exposed to noise, ABR measurements are determined prior to and 10 days after noise exposure. The mice are anesthetized with an intramuscular injection of 100 mg of ketamine and 5 mg of xylazine/kg and kept warm with a heating pad. The positive needle electrode is sub-dermally inserted at the vertex, the midline of the scalp between the external auditory canals. The negative electrode is placed below the pinna of the left ear, and the ground electrode is inserted contralaterally. Tone bursts of 12 and 24 kHz (10 ms duration, 1 ms rise and fall time) are generated using a SigGen software package (Tucker-Davis Technologies, Gainsville, Fla.) and delivered to the left external auditory meatus in a closed acoustic system through an ear bar connected to a DT48 transducer (Beyer Dynamic, Farmingdale, N.Y.). Responses from 1020 stimuli are averaged for each frequency, fed to an amplifier, viewed with an oscilloscope, and recorded. Threshold is determined by reducing the sound intensity in 5 dB steps until threshold. Thresholds are defined as the lowest intensity that yielded a reproducible deflection in the evoked response trace and are verified at least twice. Threshold shifts are calculated for individual animals by comparison to their pre-study thresholds. The ABR of each animal is interpreted by an observer without knowledge of the treatment.

Hair Cell Counts

After the last ABR measurement, animals are sacrificed, and the temporal bones are removed. The round and oval windows and the apex of the cochlea are opened, perfused with 4% paraformaldehyde in 10 mM phosphate-buffered saline (pH 7.4), and fixed overnight at 4° C. The fixed cochleae are decalcified with 4% EDTA in 10 mM phosphate-buffered saline for 2 to 3 days. After removal of the bony capsule, lateral wall, and tectorial membrane, cochleae are stained with rhodamine phalloidin for 50 min to outline the hair cells. Thereafter, the organ of Corti is separated from the modiolus, microdissected into individual turns, mounted on glass slides in antifade fluorescent mounting media, and coverslipped.

Hair cells in the organ of Corti are counted on a Leitz Orthoplan upright fluorescence microscope, using a ×50 oil immersion objective lens. Evaluation began at the apex and moved down the cochlear spiral to the base, assessing successive 0.19-mm fields. For each field, the area of observation is oriented to include the row of inner hair cells and all three rows of outer hair cells longitudinally. After counting of the entire cochlea, each row is evaluated for the presence or absence of hair cells. Manual count data are entered into a computer program. The percentage of missing hair cells for each row is calculated, utilizing normative counts from control animals as 100%, and cytocochleograms are plotted for the percentage of cell loss.

Unless defined otherwise, all technical and scientific terms used herein have the same meanings as commonly understood by one of skill in the art to which the disclosed invention belongs.

Those skilled in the art will recognize, or be able to ascertain using no more than routine experimentation, many equivalents to the specific embodiments of the invention described herein. Such equivalents are intended to be encompassed by the following claims.

We claim: Zonisamide Methods Claims

1. A method of preventing or treating a hearing disorder in a mammal, comprising administering to the mammal a therapeutic amount of a composition comprising zonisamide.

2. The method of claim 1, wherein the mammal is a primate, murine or human.

3. The method of claim 1, wherein the hearing disorder is selected from the group consisting of noise-induced hearing loss, drug-induced hearing loss, central auditory hearing disorder (CAPD), tinnitus and presbyacusis.

4. The method of claim 1, wherein the composition consists essentially of zonisamide in admixture with one or more physiologically acceptable excipients.

5. The method of claim 1, wherein the composition further comprises one or more antioxidants, spin-trapping agents, or both.

6. The method of claim 1, wherein the composition further comprises one or more antioxidants.

7. The method of claim 1, wherein the composition further comprises one or more compound having norepinephrine reuptake inhibiting and serotonin reuptake inhibiting activity.

8. The method of claim 7, wherein at least one compound having norepinephrine reuptake inhibiting and serotonin reuptake inhibiting activity is selected from the group consisting of milnacipran and bicifadine.

9. The method of claim 1, wherein the composition further comprises one or more members selected from the antioxidants selected from allopurinol, glutathione, methionine, L-carnitine and mixtures of two or more thereof.

10. The method of claim 9, wherein antioxidant is allopurinol.

11. The method of claim 9, wherein the antioxidant is glutathione.

12. The method of claim 9, wherein the antioxidant is methionine.

13. The method of claim 9, wherein the antioxidant is L-carnitine.

14. The method of claim 9, wherein the antioxidant is a mixture of two or more of allopurinol, glutathione, methionine and L-carnitine.

15. The method of claim 1, wherein the composition further comprises one or more agents that bind to or metabolize reactive oxygen species and provide protection against the damage induced by toxic oxygen mediator species.

16. The method of claim 1, wherein the composition comprises zonisamide and at least one N-methyl-D-aspartate antagonist.

17. The method of claim 1, wherein the composition comprises zonisamide and at least one N-methyl-D-aspartate antagonist selected from the group consisting of magnesium, riluzole, caroverine, memantine and mixtures thereof.

18. The method of claim 1, wherein the composition comprises zonisamide in combination with riluzole.

19. The method of claim 1, wherein the composition comprises zonisamide in combination with caroverine.

20. The method of claim 1, wherein the composition comprises zonisamide in combination with memantine.

21. The method of claim 1, wherein the composition comprises zonisamide in combination with magnesium.

22. The method of claim 19, wherein at least one of the NMDA antagonists is selected from the group consisting of magnesium, riluzole, caroverine and memantine.

23. The method of claim 1, wherein the composition further comprises an agent that blocks the excitotoxic actions of glutamate within the inner ear, whereby the noise-induced damage mediating effects of glutamate are blocked, and protection of the hair cells within the cochlea of the inner ear is effected.

24. The method of claim 1, wherein the composition further comprises a means for selective serotonin reuptake inhibitor activity and a means for N-methyl-D-aspartate antagonist activity.

25. The method of claim 1, wherein the composition further comprises a compound having selective serotonin reuptake inhibitor activity and N-methyl-D-aspartate antagonist activity.

26. The method of claim 1, wherein the composition further comprises a compound having selective serotonin reuptake inhibitor activity and a compound having N-methyl-D-aspartate antagonist activity.

27. The method of claim 1, wherein the composition further comprises a selective serotonin reuptake inhibitor selected from fluoxetine, sertraline, S-citalopram and mixtures thereof and a N-methyl-D-aspartate antagonist selected from magnesium, riluzole, caroverine, memantine and mixtures thereof.

28. The method of claim 1, wherein the composition further comprises a first compound that enhances the synaptic levels of serotonin in the brain and enhances hearing, thereby improving auditory processing, increasing the signal: noise ratio of environmental sounds or heightening attention; and a second compound that blocks the excitotoxic actions of glutamate in the inner ear, thereby blocking the glutamate-mediated noise-induced damage to the hair cells of the inner ear.

29. The method of claim 1, further comprising administering to the mammal one other active compound.

30. The method of claim 29, wherein zonisamide and the other active compound are administered in the same dose.

31. The method of claim 29, wherein zonisamide and the other active compound are administered separately and substantially simultaneously.

32. The method of claim 29, wherein zonisamide and the other active compound are administered separately and at substantially different times.

33. The method of claim 1, comprising administering to the mammal zonisamide and an antioxidant.

34. The method of claim 1, comprising administering to the mammal zonisamide and an antioxidant selected from allopurinol, glutathione, methionine, L-carnitine and mixtures thereof.

35. The method of claim 34, wherein the zonisamide and the antioxidant are administered in the same dose.

36. The method of claim 34, wherein the zonisamide and the antioxidant are administered at substantially different times.

37. The method of claim 1, comprising administering to the mammal zonisamide and at least one compound having norepinephrine reuptake inhibiting and serotonin reuptake inhibiting activity.

38. The method of claim 37, wherein at least one compound having norepinephrine reuptake inhibiting and serotonin reuptake inhibiting activity is selected from the group consisting of milnacipran and bicifadine.

39. The method of claim 1, comprising administering to the mammal zonisamide and at least one other active compound that binds to or metabolizes reactive oxygen species and provides protection against oxygen species-induced damage to the hair cells of the cochlea of the inner ear.

40. The method of claim 39, wherein zonisamide and at least one other active compound are administered in the same dose.

41. The method of claim 39, wherein zonisamide and at least one other active compound are administered at substantially different times.

42. The method of claim 1, comprising administering zonisamide and at least one N-methyl-D-aspartate antagonist.

43. The method of claim 1, comprising administering zonisamide and at least one N-methyl-D-aspartate antagonist selected from the group consisting of magnesium, riluzole, caroverine, memantine and mixtures thereof.

44. The method of claim 42, wherein the zonisamide and at least one N-methyl-D-aspartate antagonist are administered in the same dose.

45. The method of claim 42, wherein the zonisamide and at least one N-methyl-D-aspartate antagonist are administered at substantially different times.

46. The method of claim 1, comprising administering zonisamide and at least one additional active compound, wherein the additional active compound blocks the excitotoxic actions of glutamate within the inner ear, thereby blocking the glutamate-mediated noise-induced damage to the hair cells of the cochlea of the inner ear.

47. The method of claim 46, wherein the zonisamide and the additional active compound are administered in the same dose.

48. The method of claim 46, wherein the zonisamide and the additional active compound are administered at substantially different times.

49. The method of claim 1, comprising administering to the mammal zonisamide and at least one additional active compound, wherein at least one additional active compound has selective serotonin reuptake inhibitory and N-methyl-D-aspartate agonist activity.

50. The method of claim 49, wherein zonisamide and at least one additional active compound are administered in the same dose.

51. The method of claim 49, wherein zonisamide and at least one additional active compound are administered at substantially different times.

52. The method of claim 1, comprising administering to the mammal zonisamide and at least one selective serotonin reuptake inhibitor and at least one N-methyl-D-aspartate antagonist.

53. The method of claim 52, wherein the selective serotonin reuptake inhibitor is selected from the group consisting of fluoxetine, sertraline, S-citalopram and mixtures of two or more thereof.

54. The method of claim 52 or 53, wherein the N-methyl-D-aspartate antagonist is selected from the group consisting of magnesium, riluzole, caroverine, memantine and mixtures thereof.

55. The method of claim 1, further comprising administering to the mammal zonisamide, a compound that enhances the synaptic levels of serotonin in the brain and enhancing hearing, thereby improving auditory processing, increasing the signal: noise ratio of environmental sounds or heightening attention and a compound that blocks the excitotoxic actions of glutamate in the inner ear, thereby blocking the glutamate-mediated noise-induced damage to the hair cells of the inner ear
Zonisamide Composition Claims

56. A composition for treating or preventing hearing loss in a mammal comprising a therapeutic amount of zonisamide.

57. The composition of claim 56, wherein the mammal is a primate, murine or human.

58. The composition of claim 57, wherein the mammal is a human.

59. The composition of claim 56, wherein the composition consists essentially of zonisamide in admixture with one or more physiologically acceptable excipients.

60. The composition of claim 56, wherein the composition further comprises one or more antioxidants, spin-trapping agents, or both.

61. The composition of claim 56, wherein the composition further comprises one or more antioxidants.

62. The composition of claim 56, wherein the composition further comprises one or more antioxidants selected from allopurinol, glutathione, methionine, L-carnitine and mixtures of two or more thereof.

63. The composition of claim 62, wherein antioxidant is allopurinol.

64. The composition of claim 62, wherein the antioxidant is glutathione.

65. The composition of claim 62, wherein the antioxidant is methionine.

66. The composition of claim 62, wherein the antioxidant is L-carnitine.

67. The composition of claim 62, wherein the antioxidant is a mixture of two or more of allopurinol, glutathione, methionine and L-carnitine.

68. The composition of claim 56, wherein the composition further comprises one or more agents that bind to or metabolize reactive oxygen species and provide protection against the damage induced by toxic oxygen mediator species.

69. The composition of claim 56, wherein the composition comprises zonisamide and at least one NMDA antagonist.

70. The composition of claim 56, wherein the composition comprises zonisamide and at least one NMDA antagonist selected from the group consisting of magnesium, riluzole, caroverine, memantine and mixtures thereof.

71. The composition of claim 56, wherein the composition comprises zonisamide in combination with riluzole.

72. The composition of claim 56, wherein the composition comprises zonisamide in combination with caroverine.

73. The composition of claim 56, wherein the composition comprises zonisamide in combination with memantine.

74. The composition of claim 56, wherein the composition comprises zonisamide in combination with magnesium.

75. The composition of claim 74, wherein at least one of the NMDA antagonists is selected from the group consisting of magnesium, riluzole, caroverine and memantine.

76. The composition of claim 56, wherein the composition further comprises an agent that blocks the excitotoxic actions of glutamate within the inner ear, whereby the noise-induced damage mediating effects of glutamate are blocked, and protection of the hair cells within the cochlea of the inner ear is effected.

77. The composition of claim 56, wherein the composition further comprises a means for selective serotonin reuptake inhibitor activity and a means for N-methyl-D-aspartate antagonist activity.

78. The composition of claim 56, wherein the composition further comprises a compound having selective serotonin reuptake inhibitor activity and N-methyl-D-aspartate antagonist activity.

79. The composition of claim 56, wherein the composition further comprises a compound having selective serotonin reuptake inhibitor activity and a compound having N-methyl-D-aspartate antagonist activity.

80. The composition of claim 56, wherein the composition further comprises a selective serotonin reuptake inhibitor selected from fluoxetine, sertraline, S-citalopram and mixtures thereof and a N-methyl-D-aspartate antagonist selected from magnesium, riluzole, caroverine, memantine and mixtures thereof.

81. The composition of claim 56, wherein the composition further comprises a means for enhancing the synaptic levels of serotonin in the brain and enhancing hearing, thereby improving auditory processing, increasing the signal: noise ratio of environmental sounds or heightening attention and a means for blocking the excitotoxic actions of glutamate in the inner ear, thereby blocking the glutamate-mediated noise-induced damage to the hair cells of the inner ear.

82. The composition of claim 56, wherein the composition further comprises one or more compounds having norepinephrine reuptake inhibiting and serotonin reuptake inhibiting activity.

83. The composition of claim 82, wherein at least one compound having norepinephrine reuptake inhibiting and serotonin reuptake inhibiting activity is selected from the group consisting of milnacipran and bicifadine.

84. A kit for treating or preventing hearing loss in a mammal, comprising zonisamide and at least one other active pharmaceutical ingredient.

85. The kit of claim 84, wherein zonisamide and the other active compound are compounded in the same dose.

86. The kit of claim 84, wherein zonisamide and the other active compound are compound in separate doses.

87. The kit of claim 84, comprising zonisamide and an antioxidant.

88. The kit of claim 84, comprising zonisamide and an antioxidant selected from allopurinol, glutathione, methionine, L-carnitine and mixtures thereof.

89. The kit of claim 88, wherein the zonisamide and the antioxidant are compounded in the same dose.

90. The kit of claim 88, wherein the zonisamide and the antioxidant are compounded in separate doses.

91. The kit of claim 84, comprising zonisamide and at least one other active compound, wherein at least one other active compound binds to or metabolizes reactive oxygen species and provides protection against oxygen species-induced damage to the hair cells of the cochlea of the inner ear.

92. The kit of claim 91, wherein zonisamide and at least one other active compound are kit compound in the same dose.

93. The kit of claim 84, wherein zonisamide and at least one other active compound are compounded in separate doses.

94. The kit of claim 84, comprising zonisamide and at least one N-methyl-D-aspartate antagonist.

95. The kit of claim 84, comprising zonisamide and at least one N-methyl-D-aspartate antagonist selected from the group consisting of magnesium, riluzole, caroverine, memantine and mixtures thereof.

96. The kit of claim 95, wherein the zonisamide and at least one N-methyl-D-aspartate antagonist are compounded in the same dose.

97. The kit of claim 95, wherein the zonisamide and at least one N-methyl-D-aspartate antagonist are compounded in separate doses.

98. The kit of claim 81, comprising zonisamide and at least one compound having selective serotonin reuptake inhibitory and N-methyl-D-aspartate agonist activity.

99. The kit of claim 98, wherein zonisamide and at least one additional active compound are compounded in the same dose.

100. The kit of claim 99, wherein zonisamide and at least one additional active compound are compounded in separate doses.

101. The kit of claim 84, comprising zonisamide and at least one selective serotonin reuptake inhibitor and at least one N-methyl-D-aspartate antagonist.

102. The kit of claim 101, wherein the selective serotonin reuptake inhibitor is selected from the group consisting of fluoxetine, sertraline, S-citalopram and mixtures of two or more thereof.

103. The kit of claim 101 or 102, wherein the N-methyl-D-aspartate antagonist is selected from the group consisting of magnesium, riluzole, caroverine, memantine and mixtures thereof.

104. The kit of claim 84, comprising a first compound that enhances the synaptic levels of serotonin in the brain and enhances hearing, thereby improving auditory processing, increasing the signal: noise ratio of environmental sounds or heightening attention; and a second compound that blocks the excitotoxic actions of glutamate in the inner ear, thereby blocking the glutamate-mediated noise-induced damage to the hair cells of the inner ear.

105. The kit of claim 84, comprising a zonisamide and having norepinephrine reuptake inhibiting and serotonin reuptake inhibiting activity.

106. The kit of claim 105, wherein at least one compound having norepinephrine reuptake inhibiting and serotonin reuptake inhibiting activity is selected from the group consisting of milnacipran and bicifadine.

107. The kit of claim 105 or 106, wherein the zonisamide and at least one compound having norepinephrine reuptake inhibiting and serotonin reuptake inhibiting activity are in separate doses.

108. The kit of claim 105 or 106, wherein the zonisamide and at least one compound having norepinephrine reuptake inhibiting and serotonin reuptake inhibiting activity are in the same dose.
Amantadine Claims

109. A method of preventing or treating a hearing disorder in a mammal, comprising administering to the mammal a therapeutic amount of a composition comprising a compound having dopamine releasing and NMDA antagonist activity.

110. The method of claim 109, wherein the compound having dopamine releasing and NMDA antagonist activity is amantadine.

111. The method of claim 109 or 110, further comprising administering to the mammal a one or more additional compounds selected from the group consisting of zonisamide, selective serotonin reuptake inhibitors and antioxidants.

112. The method of claim 111, wherein at least one said additional compound is compounded with amantadine in the same dosage form.

113. The method of claim 112, wherein the dosage form is an oral dosage form.

114. The method of claim 111, wherein amantadine and at least one said additional compound are compounded in separate dosage forms.

115. The method of claim 114, wherein amantadine and at least one said additional compound are administered at separate times.

116. The method of claim 110, wherein the amantadine is in an oral dosage form.

117. A composition for the prevention or treatment of a hearing disorder, comprising an effective amount of a compound having dopamine releasing and NMDA antagonist activity.

118. The composition of claim 117, wherein the compound having dopamine releasing and NMDA antagonist activity is amantadine.

119. The composition of claim 117 or 118, further comprising one or more compounds selected from the group consisting of zonisamide, selective serotonin reuptake inhibitors and antioxidants.

120. A dosage form for the prevention or treatment of a hearing disorder, comprising an effective amount of a compound having dopamine releasing and NMDA antagonist activity and one or more additional ingredients.

121. The dosage form of claim 120, wherein the compound having dopamine releasing and NMDA antagonist activity is amantadine.

122. The dosage form of claim 120 or 121, wherein at least one additional ingredient is selected from the group consisting of zonisamide, selective serotonin reuptake inhibitors and antioxidants.

123. The dosage form of claim 120 or 121, comprising at least one inactive ingredient.

124. The dosage form of claim 120 or 121, comprising at least one inactive ingredient and at least one ingredient selected from the group consisting of zonisamide, selective serotonin reuptake inhibitors and antioxidants.

125. A kit comprising the dosage form of claim 120 or 121 and at least one dosage form comprising a second active ingredient selected from the group consisting of zonisamide, selective serotonin reuptake inhibitors and antioxidants.

126. The kit of claim 125, further comprising dosing instructions.

127. The method of claim 109, 120 or 121, wherein the hearing disorder is selected from the group consisting of noise-induced hearing loss, drug-induced hearing loss, central auditory hearing disorder (CAPD), tinnitus and presbyacusis.
Bifemelane Claims

128. A method of preventing or treating a hearing disorder in a mammal, comprising administering to the mammal a therapeutic amount of a composition comprising a compound having acetylcholine release inducing, antioxidant, NMDA antagonist and norepinephrine reuptake inhibiting activity.

129. The method of claim 128, wherein compound having acetylcholine release inducing, antioxidant, NMDA antagonist and norepinephrine reuptake inhibiting activity is bifemelane.

130. The method of claim 128 or 129, wherein the hearing disorder is selected from the group consisting of noise-induced hearing loss, drug-induced hearing loss, central auditory hearing disorder (CAPD), tinnitus and presbyacusis.

131. A composition for the prevention or treatment of a hearing disorder, comprising an effective amount of a compound having acetylcholine release inducing, antioxidant, NMDA antagonist and norepinephrine reuptake inhibiting activity.

132. The composition of claim 131, wherein the compound having dopamine releasing and NMDA antagonist activity is bifemelane.

133. A dosage form for the prevention or treatment of a hearing disorder, comprising an effective amount of a compound having acetylcholine release inducing, antioxidant, NMDA antagonist and norepinephrine reuptake inhibiting activity.

134. The dosage form of claim 133, wherein the compound having dopamine releasing and NMDA antagonist activity is bifemelane.

135. The dosage form of claim 133 or 134, comprising at least one inactive ingredient.
Pirlindose Claims

136. A method of preventing or treating a hearing disorder in a mammal, comprising administering to the mammal a therapeutic amount of a composition comprising a compound having MAO-A inhibiting, serotonin reuptake inhibiting and antioxidant activity.

137. The method of claim 136, wherein compound having MAO-A inhibiting, serotonin reuptake inhibiting and antioxidant activity is pirlindole.

138. The method of claim 136 or 137, wherein at least one said additional compound is compounded with bifemelane in the same dosage form.

139. The method of claim 136 or 137, wherein the hearing disorder is selected from the group consisting of noise-induced hearing loss, drug-induced hearing loss, central auditory hearing disorder (CAPD), tinnitus and presbyacusis.

140. A composition for the prevention or treatment of a hearing disorder, comprising an effective amount of a compound having MAO-A inhibiting, serotonin reuptake inhibiting and antioxidant activity.

141. The composition of claim 140, wherein the compound having MAO-A inhibiting, serotonin reuptake inhibiting and antioxidant activity is pirlindole.

142. A dosage form for the prevention or treatment of a hearing disorder, comprising an effective amount of a compound having MAO-A inhibiting, serotonin reuptake inhibiting and antioxidant activity.

143. The dosage form of claim 142, wherein the compound having dopamine releasing and NMDA antagonist activity is pirlindole.

144. The dosage form of claim 142 or 143, comprising at least one inactive ingredient.
Calcium Channel Blocker Claims

145. A method of preventing or treating a hearing disorder in a mammal, comprising administering to the mammal a therapeutic amount of a composition comprising at least a first compound having calcium channel blocking activity and a second compound having selective serotonin reuptake inhibiting activity, norepinephrine-serotonin reuptake inhibiting activity or monamineoxidase-A inhibiting activity.

146. The method of claim 145, wherein the first compound is selected from nimodipine and verapamil.

147. The method of claim 145, wherein the first compound and the second compound are combined in a single dosage form.

148. The method of claim 147, wherein the first compound is selected from nimodipine and verapamil.

149. The method of claim 147 or 148, wherein the dosage form is an oral dosage form.

150. The method of claim 145, wherein the first compound and the second compound are administered simultaneously.

151. The method of claim 145, wherein the first compound and the second compound are administered at separate times.

152. A composition for the prevention or treatment of a hearing disorder, comprising an effective amount of a first compound having calcium channel blocking activity and a second compound having selective serotonin reuptake inhibiting activity, norepinephrine-serotonin reuptake inhibiting activity or monamineoxidase-A inhibiting activity.

153. The composition of claim 152, wherein the first compound is nimodipine or verapamil.

154. A dosage form for the prevention or treatment of a hearing disorder, comprising an effective amount of a first compound having calcium channel blocking activity and a second compound having selective serotonin reuptake inhibiting activity, norepinephrine-serotonin reuptake inhibiting activity or monamineoxidase-A inhibiting activity.

155. The dosage form of claim 154, wherein the first compound is nimodipine or verapamil.

156. The dosage form of claim 154 or 155, comprising at least one inactive ingredient.

157. A kit comprising a first dosage form comprising a calcium channel antagonist and a second dosage form comprising a serotonin reuptake inhibitor, a norepinephrine-serotonin reuptake inhibitor or a monamineoxidase-A inhibitor.

158. The kit of claim 157, wherein the calcium channel antagonist is selected from the group consisting of nimodipine, verapamil or a combination thereof.

159. The method of claim 145, wherein the hearing disorder is selected from the group consisting of noise-induced hearing loss, drug-induced hearing loss, central auditory hearing disorder (CAPD), tinnitus and presbyacusis.
Indeloxazine Claims

160. A method of preventing or treating a hearing disorder in a mammal, comprising administering to the mammal a therapeutic amount of a composition comprising at least a first compound having 5HT reuptake inhibiting, norepinephrine reuptake inhibiting, acetylcholine releasing and N-methyl-D-aspartate antagonistic activity.

161. The method of claim 160, wherein the first compound is indeloxazine.

162. The method of claim 160 or 161, further comprising administering to the mammal a second compound having N-methyl-D-aspartate antagonistic activity.

163. The method of claim 162, wherein the first compound and the second compound are combined in a single dosage form.

164. The method of claim 163, wherein the dosage form is an oral dosage form.

165. The method of claim 162, wherein the first compound and the second compound are administered simultaneously.

166. The method of claim 162, wherein the first compound and the second compound are administered at separate times.

167. A composition for the prevention or treatment of a hearing disorder, comprising an effective amount of at least a first compound having 5HT reuptake inhibiting, norepinephrine reuptake inhibiting, acetylcholine releasing and N-methyl-D-aspartate antagonistic activity.

168. The composition of claim 167, wherein the first compound is indeloxazine.

169. The composition of claim 167 or 168, wherein the composition further comprises a second compound having N-methyl-D-aspartate antagonistic activity.

170. A dosage form for the prevention or treatment of a hearing disorder, comprising an effective amount of at least a first compound having 5HT reuptake inhibiting, norepinephrine reuptake inhibiting, acetylcholine releasing and N-methyl-D-aspartate antagonistic activity.

171. The dosage form of claim 170, wherein the first compound is indeloxazine.

172. The dosage form of claim 170 or 171, further comprising a second compound having N-methyl-D-aspartate antagonistic activity.

173. The dosage form of claim 172, further comprising at least one inactive ingredient.

174. A kit comprising a first dosage form comprising a first compound having 5HT reuptake inhibiting, norepinephrine reuptake inhibiting, acetylcholine releasing and N-methyl-D-aspartate antagonistic activity and a second dosage form comprising a second compound having N-methyl-D-aspartate antagonistic activity.

175. The kit of claim 172, wherein the first compound is indeloxazine.

176. The method of claim 170, wherein the hearing disorder is selected from the group consisting of noise-induced hearing loss, drug-induced hearing loss, central auditory hearing disorder (CAPD), tinnitus and presbyacusis.
Methods of Treating CAPD

177. A method of treating central auditory processing disorder, comprising administering to a patient a therapeutically effective amount of a therapeutic composition comprising at least one compound selected from the group consisting of: zonisamide; compounds having antioxidants activity; compounds having N-methyl-D-aspartate (NMDA) antagonist activity; selective serotonin reuptake inhibitors; compounds having dopamine releaser and NMDA antagonist activity; combinations of at least one dopamine releaser and at least one NMDA antagonist; compounds having acetycholine release inducer, antioxidant, NMDA antagonist and norepinephrine-epinephrine reuptake inhibitor (NERI) activity; compounds having monamine oxidase A inhibitor, serotonin reuptake inhibitor and antioxidant activity; compounds having norepinephrine and serotonin reuptake inhibitor and low affinity NMDA antagonist activity; calcium channel antagonists; norepinephrine selective reuptake inhibitors (NSRIs); compounds having 5HT selective serotonin reuptake inhibitor, norepinephrine selective reuptake inhibitor, acetycholine releaser and NMDA antagonist activity; and pharmaceutically acceptable salts and polymorphs thereof.

178. The method of claim 177, wherein the therapeutic composition comprises a selective serotonin reuptake inhibitor compound or a pharmaceutically acceptable salt or polymorph thereof.

179. The method of claim 177, wherein the selective serotonin reuptake inhibitor compound is selected from the group consisting of fluoxetine, sertraline, S-citalopram and alaproclate.

180. The method of claim 177, wherein the therapeutic composition comprises a norepinephrine reuptake inhibitor compound or a pharmaceutically acceptable salt or polymorph thereof.

181. The method of claim 180, wherein the norepinephrine reuptake inhibitor compound is selected from the group consisting of NERIs, NRIs and NSRIs.

182. The method of claim 180, wherein the norepinephrine reuptake inhibitor compound is selected from the group consisting of bifemelane, indeloxazine, atomoxetine, milnacipran and bicifadine.

183. The method of claim 177, wherein the therapeutic composition comprises a compound having acetylcholinesterase inducer activity, or a pharmaceutically acceptable salt or polymorph thereof.

184. The method of claim 185, wherein the compound having acetylcholinesterase inducer activity is bifemelane.

185. The method of claim 177, wherein the therapeutic composition comprises at least two compounds selected from the group consisting of: zonisamide; compounds having antioxidants activity; compounds having N-methyl-D-aspartate (NMDA) antagonist activity; selective serotonin reuptake inhibitors; compounds having dopamine releaser and NMDA antagonist activity; combinations of at least one dopamine releaser and at least one NMDA antagonist; compounds having acetycholine release inducer, antioxidant, NMDA antagonist and norepinephrine-epinephrine reuptake inhibitor (NERI) activity; compounds having monamine oxidase A inhibitor, serotonin reuptake inhibitor and antioxidant activity; compounds having norepinephrine and serotonin reuptake inhibitor and low affinity NMDA antagonist activity; calcium channel antagonists; norepinephrine selective reuptake inhibitors (NSRIs); compounds having 5HT selective serotonin reuptake inhibitor, norepinephrine selective reuptake inhibitor, acetycholine releaser and NMDA antagonist activity; and pharmaceutically acceptable salts and polymorphs thereof.

186. The method of claim 185, wherein the therapeutic composition comprises a combination of zonisamide, or a pharmaceutically acceptable salt thereof, and a compound, or pharmaceutically acceptable salt or polymorph thereof, having norepinephrine-epinephrine reuptake inhibitor activity.

187. The method of claim 185, wherein the therapeutic composition comprises a combination of zonisamide, or a pharmaceutically acceptable salt thereof, and a compound, or pharmaceutically acceptable salt or polymorph thereof, having norepinephrine and serotonin reuptake inhibitor and low-affinity NMDA antagonist activity.

188. The method of claim 185, wherein the therapeutic composition comprises a combination of two or more antioxidants.

189. The method of claim 185, wherein the therapeutic composition comprises a combination of two or more NMDA antagonists.

190. The method of claim 185, wherein the therapeutic composition comprises a combination of a SSRI and a NMDA antagonist.

191. The method of claim 185, wherein the therapeutic composition comprises a combination of a dopamine releaser and a NMDA antagonist.

192. The method of claim 185, wherein the therapeutic composition comprises a combination of a calcium channel antagonist and a member selected from the group consisting of SSRIs and NSRIs.

193. The method of claim 185, wherein the therapeutic composition comprises a combination of a first compound, or pharmaceutically acceptable salt or polymorph thereof, having 5HT serotonin reuptake inhibitor, norepinephrine reuptake inhibitor, acetylcholine releaser and N-methyl-D-aspartate antagonist activity with a second compound, or pharmaceutically acceptable salt or polymorph thereof, having NMDA antagonist activity.

194. The method of claim 185, wherein the first compound is indeloxazine.

195. The method of claim 185, wherein the therapeutic composition comprises at least one member of the group consisting of: zonisamide, allopurinol, glutathione, methionine and L-carnitine, riluzole, caroverine, memantine, magnesium, include fluoxetine, sertraline, S-citalopram, amantadine, bifemelane, pirlindole milnacipran, bicifadine, nimodipine, verapamil, atomoxetine, indeloxazine; and pharmaceutically acceptable salts and polymorphs thereof.

196. The method of claim 195, wherein the therapeutic composition comprises at least two members of the group consisting of: zonisamide, allopurinol, glutathione, methionine and L-carnitine, riluzole, caroverine, memantine, magnesium, include fluoxetine, sertraline, S-citalopram, amantadine, bifemelane, pirlindole milnacipran, bicifadine, nimodipine, verapamil, atomoxetine, indeloxazine; and pharmaceutically acceptable salts and polymorphs thereof.

197. The method of claim 196, wherein the therapeutic composition comprises two or more compounds, or pharmaceutically acceptable salts or polymorphs thereof, selected from the group consisting of allopurinol, glutathione, methionine and L-carnitine.

198. The method of claim 196, wherein the therapeutic composition comprises two or more NMDA antagonist compounds, or pharmaceutically acceptable salts or polymorphs thereof, selected from the group consisting of: riluzole, caroverine, memantine and magnesium.

199. The method of claim 196, wherein the therapeutic composition comprises at least one SSRI compound, or a pharmaceutically acceptable salt or polymorph thereof, and at least one NMDA antagonist compound, or a pharmaceutically acceptable salt thereof, wherein the SSRI compound is selected from the group consisting of: fluoxetine, sertraline, S-citalopram, alaproclate and the NMDA antagonist compound is selected from the group consisting of: riluzole, caroverine, memantine and magnesium.

200. The method of claim 196, wherein the therapeutic composition comprises amantadine or a pharmaceutically acceptable salt or polymorph thereof and at least one additional compound, or a pharmaceutically acceptable salt or polymorph thereof, wherein the additional compound is selected from the group consisting of: zonisamide, selective serotonin reuptake inhibitors, and antioxidants.

201. The method of claim 200, wherein the therapeutic composition comprises: amantadine; a selective serotonin reuptake inhibitor compound or therapeutically acceptable salt or polymorph thereof; and an antioxidant compound or pharmaceutically acceptable salt or polymorph thereof.

202. The method of claim 200 or 201, wherein the therapeutic composition comprises zonisamide or a pharmaceutically acceptable salt or polymorph thereof.

203. The method of claim 196, wherein the therapeutic composition comprises zonisamide or a pharmaceutically acceptable salt or polymorph thereof and at least one additional compound, or a pharmaceutically acceptable salt or polymorph thereof, wherein the additional compound is selected from the group consisting of: amantadine, selective serotonin reuptake inhibitors, and antioxidants.

204. The method of claim 203, wherein the therapeutic composition comprises: zonisamide; a selective serotonin reuptake inhibitor compound or therapeutically acceptable salt or polymorph thereof, and an antioxidant compound or pharmaceutically acceptable salt or polymorph thereof.

205. The method of claim 202 or 203, wherein the therapeutic composition comprises amantadine or a pharmaceutically acceptable salt or polymorph thereof.

206. The method of claim 196, wherein the therapeutic composition comprises a calcium channel antagonist or pharmaceutically acceptable salt or polymorph thereof and a SSRI compound or pharmaceutically acceptable salt or polymorph thereof, wherein the calcium channel antagonist is selected from the group consisting of nimodipine, verapamil and combinations thereof.

207. The method of claim 206, wherein the SSRI compound is selected from the group consisting of fluoxetine, sertraline, S-citalopram and alaproclate.

208. The method of claim 196, wherein the therapeutic composition comprises a calcium channel antagonist or pharmaceutically acceptable salt or polymorph thereof and a NMDA antagonist compound or pharmaceutically acceptable salt or polymorph thereof; wherein the calcium channel antagonist is selected from the group consisting of nimodipine, verapamil and combinations thereof.

209. The method of claim 208, wherein the NMDA antagonist compound is selected from the group consisting of riluzole, caroverine, memantine and magnesium.

210. The method of claim 196, wherein the therapeutic composition comprises indeloxazine, or a pharmaceutically acceptable salt or polymorph thereof, and at least one NMDA antagonist compound or a pharmaceutically acceptable salt or polymorph thereof.

211. The method of claim 210, wherein the NMDA antagonist compound is selected from the group consisting of riluzole, caroverine, memantine and magnesium.
